SEQUENCE LISTING (18 sequences)

SEQ ID NO: 1 - protein, 362 aa, Homo sp.
KISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 2 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 3 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 4 - protein, 349 aa, synthetic construct
KISQYAAQRRTTLNNYNKKFTDALDILAENAXLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 5 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLXSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 6 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSXVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 7 - protein, 349 aa, synthetic construct
KISQYAAQRRTTLNNYNKKFTDALDILAENACLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 8 - protein, 361 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERN

SEQ ID NO: 9 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLCSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 10 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSCVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 11 - protein, 362 aa, synthetic construct
KISQYAAQRRTTLNNYNKKFTDALDILAENACLRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 12 - protein, 362 aa, synthetic construct
KISQYAAQRRTTLNNYNKKFTDALDILACNAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 13 - protein, 362 aa, synthetic construct
KISQYAAQRRTTLNNYNKKFTDALDILAENAELRGNCGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 14 - protein, 362 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLHHQRVDSGKSGQQEGKGWKAIRVDLVMAPYERRAFALLGWTGSRQFERDLRRYARHERKMLLDNHALYDRTKNTFLRAESEEEIFAHLGLEYIEPWERNA

SEQ ID NO: 15 - protein, 349 aa, synthetic construct
KISQYAAQRRTTLNNYNKKFTDALDILACNAELRGNEGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 16 - protein, 349 aa, synthetic construct
KISQYAAQRRTTLNNYNKKFTDALDILAENAELRGNCGSALAFRRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 17 - protein, 349 aa, synthetic construct
KISQYAAQRRTTLNNYNKKFTDALDILAENAELRGNEGSALAFCRAASVLKSLPFPITSMKDTEGIPNLGDKVKRIIQEILEDGESSEAKAVLNDERYQAFKLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

SEQ ID NO: 18 - protein, 349 aa, synthetic construct
KISQYAAQRRTTLNNYNKKFTDALDILAENAELRGNEGSALAFRRAASVLKSLPFPITSMKDTEGIPNLGDKVKRIIQEILEDGESSEAKAVLNDERYQAFCLFTSVFGVGPKTAEKWYRMGFRTLEDIRSDKTLRFTRMQKAGFLYYEDLVSPVTRPEAEAIEQLVKEAVWQFLPGATVTMTGGFRRGKKTGHDVDFLITSPEATEEEEKQLLHKVTDWWKKQGLLLYEDIQESTFEKFKLPSRKVDALDHFQKAFLILKLGPSPGKAIRVDLVMAPYERRAFALLGWTGSRQFERDLRRYARHERKMLLDNHALYDRTKNTFLRAESEEEIFAHLGLEYIEPWERNA

(19) United States
(12) Patent Application Publication
(10) Pub. No.: US 2025/0154548 A1
(43) Pub. Date: May 15, 2025

(54) CONTROL OF ENZYMATIC NUCLEIC ACID SYNTHESIS VIA ELECTROCHEMICAL MEANS

(71) Applicant: The Charles Stark Draper Laboratory, Inc., Cambridge, MA (US)

(72) Inventors: Cassie M. Bryan, Cambridge, MA (US); Melissa Sprachman, Cambridge, MA (US); Robert Doyle, Cambridge, MA (US); William C. Records, Cambridge, MA (US)

(21) Appl. No.: 18/941,957

(22) Filed: Nov. 8, 2024

Related U.S. Application Data
(60) Provisional application No. 63/597,675, filed on Nov. 9, 2023.

(51) Int. Cl. C12P 19/34; C12N 9/12; C12N 9/50

(52) U.S. Cl. CPC C12P 19/34; C12N 9/1264; C12N 9/50; C12Y 207/07031; C12Y 304/21064

(57) ABSTRACT

Provided herein are methods of nucleic acid synthesis. In some embodiments, a method of nucleic acid synthesis comprises selectively activating and deactivating an engineered terminal deoxynucleotidyl transferase (TdT). In some embodiments, a method of nucleic acid synthesis comprises activating a pH sensitive enzyme to digest and remove a TdT.

### RELATED APPLICATION(S)

This application claims the benefit of U.S. Provisional Application No. 63/597,675, filed on Nov. 9, 2023. The entire teachings of the above application are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 50001072-001_SL.xml; created Nov. 8, 2024, 22,858 bytes in size.

### BACKGROUND

DNA polymerases are enzymes responsible for the replication of genetic material in vivo and in vitro. Specifically, these enzymes are responsible for catalyzing the addition of nucleotide triphosphates (e.g., dNTPs and analogs thereof) to the three-prime end of a primer or seed strand of DNA. The majority of DNA polymerases replicate DNA in a largely template-dependent manner. That is: synthesizing the reverse complement strand of a DNA strand. However, a few polymerases have template-independent activity, wherein they can synthesize random sequences of DNA without the influence or need of a template strand.

Terminal deoxynucleotidyl transferase (hereinto referred to as TdT) is a DNA polymerase capable of catalyzing the random addition of nucleotides. In vivo, specifically in premature immune cells undergoing antibody and T cell receptor recombination, TdT acts in conjunction with DNA repair pathways to generate highly diverse sequences at VDJ junction sites. In vitro, TdT also displays template-independent activity, enabling its widespread use for applications such as poly A tailing of DNA. Because TdT does not require a DNA primer strand for DNA synthesis it can be used as an enzyme for in vitro DNA synthesis.

However, TdT adds nucleotides in an uncontrolled manner. While this template independence provides a means to synthesize entirely novel sequences of DNA from scratch, to do so in a highly controlled and sequence specific manner requires the control of the enzyme.

The availability of a TdT enzyme capable of controllably adding or inserting a single nucleotide (mononucleotide) at a time in a selective and/or controlled manner would enable new DNA synthesis strategies not previously possible, with benefits over existing strategies, and in particular would enable control of the enzyme for the synthesis of sequence-specified DNA, RNA, or other nucleic acid molecules.

### SUMMARY

In accordance with the description, a first method of nucleic acid molecule synthesis comprises
a) providing a single-stranded nucleic acid molecule comprising a 5′ and a 3′ end;
b) providing an engineered terminal deoxynucleotidyl transferase (TdT), wherein the engineered TdT comprises a nucleotide molecule covalently attached to the TdT via a cleavable linker;
c) providing at least one metal cofactor complexed to at least one cleavable chelating agent, wherein the chelating agent is capable of releasing the metal cofactor upon cleavage;
d) selectively activating the TdT by cleaving the chelating agent;
e) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand;
f) exposing the TdT to conditions sufficient to deactivate it and remove it from the nucleic acid molecule; and
g) repeating steps (b) through (f) thereby synthesizing a nucleic acid molecule.

A second method of nucleic acid molecule synthesis in a buffer comprises the steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker and at least one metal cofactor;
(c) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(d) providing an inactivated pH sensitive enzyme, wherein the pH sensitive enzyme is inactivated by pH conditions of the buffer;
(e) activating the pH sensitive enzyme through a change in pH conditions in the buffer and digesting the TdT releasing the extended nucleic acid strand;
(f) inactivating the pH sensitive enzyme through a change in pH conditions; and
(g) repeating steps (c) through (f) at least once thereby synthesizing a nucleic acid molecule.

A third method of nucleic acid molecule synthesis in a buffer comprises the steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker;
(c) providing at least one metal cofactor complexed to at least one cleavable chelating agent, wherein the chelating agent is capable of releasing the metal cofactor upon cleavage;
(d) selectively activating the TdT by cleaving the chelating agent;
(e) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(f) providing an inactivated pH sensitive enzyme, wherein the pH sensitive enzyme is inactivated by pH conditions of the buffer;
(g) activating the pH sensitive enzyme through a change in pH conditions in the buffer and digesting the TdT releasing the extended nucleic acid strand;
(h) inactivating the pH sensitive enzyme through a change in pH conditions; and
(i) repeating steps (c) through (h) at least once thereby synthesizing a nucleic acid molecule.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are illustrative and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

The foregoing will be apparent from the following more particular description of example embodiments, including those illustrated in the drawings interspersed herein. The drawings are not necessarily to scale, emphasis instead being placed upon illustrating embodiments.

FIGS. 1A-1C provide information on activating proteinase K at different pH conditions. FIG. 1A shows a schematic for activating proteinase K by applying a localized voltage to a solid surface comprising multiple sites for nucleic acid synthesis. FIG. 1B shows proteinase K activity (relative to activity at pH 7.3) as a function of pH. pH values where proteinase K's activity falls below 10% of its activity at pH 7.3 are labelled as “OFF.” FIG. 1C shows the relative activity of proteinase K at a starting pH and after neutralization to pH 7 to 7.5, showing that proteinase K recovers even after exposed to highly acidic (e.g., pH 3.5) or basic (e.g., pH 12) conditions.

FIG. 2 illustrates an example method of inducing a pH change by applying localized voltage in the presence of a proton donor/acceptor, R.

FIG. 3A shows a fluorescent microscope image of a microfabricated set of planar electrodes in the presence of an aqueous electrolyte containing 100 mM NaCl, 25 mM buffer, 0.5 mM 1,4-benzoquinone, 0.5 mM 2,5-dihydroxy-1,4-benzoquinone, and 1 mM fluorescein-PEG6-amine (FLPA, structure shown in inset). The cathode (410 μm×510 μm) contains a microarray (region of interest, ROI) of 300-nm diameter wells that cannot be optically resolved but appear as a smaller square region (˜250 μm×325 μm), made brighter by optical interference.

FIG. 3B shows the average pixel intensity in the ROI as a function of bulk electrolyte pH, baselined (i.e., zeroed) to values at pH 3.5. Data points represent the average of three technical replicates, and error bars represent one standard deviation.

FIG. 3C shows the change in intensity as a function of time with different applied voltages to the working electrode (cathode).

FIG. 4 shows an example strategy for use of cleavable chelator molecules. Molecules including a tetraacid and catechol shown interact with free metal ions (e.g., Co²⁺), sequestering the metal (i.e., the chelate metal is unavailable to be used as a metal cofactor for enzymatic activity). When the chelator is cleaved, the metal is released and available as a metal cofactor for enzymatic activity.

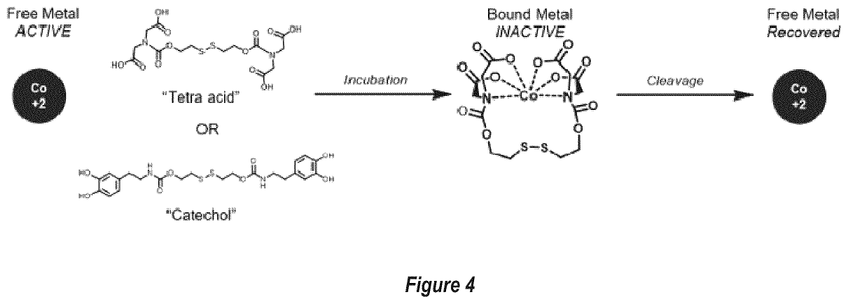

FIG. 5 shows an example of the reductive cleavage of a tetraacid chelator (also labeled as “IDA”). Molecules including a catechol, tetraacid, and dithiopyridine (“DTP”) shown interact with free metal ions (e.g., Mn²⁺), sequestering the metal (i.e., the chelate metal is unavailable to be used as a metal cofactor for enzymatic activity). When the chelator is cleaved, the metal is released and available to be a metal cofactor for enzymatic activity.

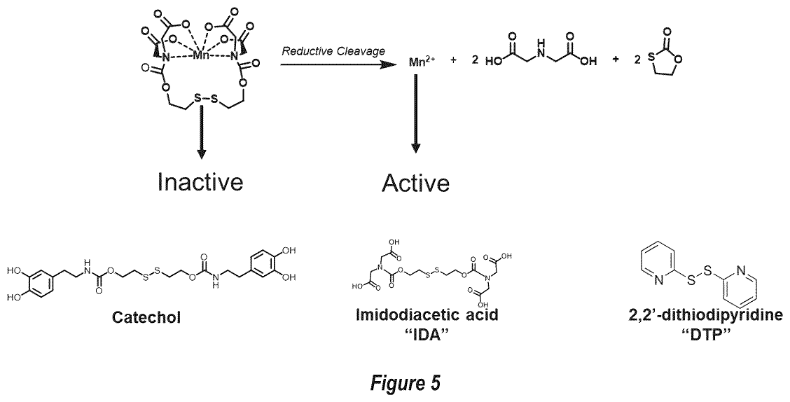

FIG. 6A shows the structure of commercially available DTP, 2,2′-dithiopyridine.

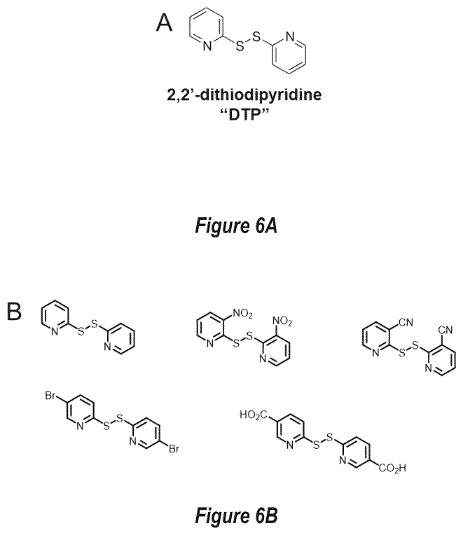

FIG. 6B shows structures of DTP analogs that are commercially available.

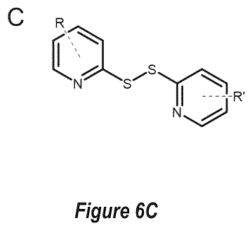

FIG. 6C shows analogs of 2,2′-dithiopyridine with R and R′ as various substituents to modulate the strength of chelation and aqueous solubility. R and R′ include but are not limited to represent any alkyl, halogen, nitro, amino, sulfo, phosphoro, alkoxy, acetyle, cyano, glycidyl, or others.

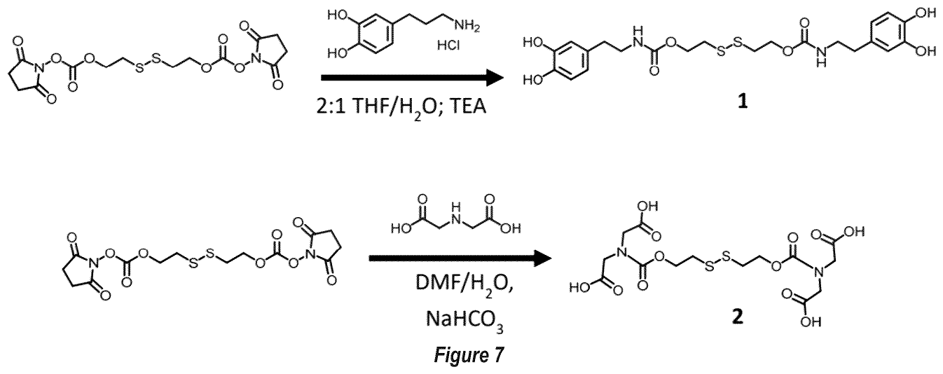

FIG. 7 shows the synthesis of cleavable chelators. Top: Synthesis of a bis-catechol with a linking, cleavable disulfide moiety; Bottom; Synthesis of a tetra acid (analog of ethylenediamine tetracetic acid, EDTA) with a linking, cleavable, disulfide moiety.

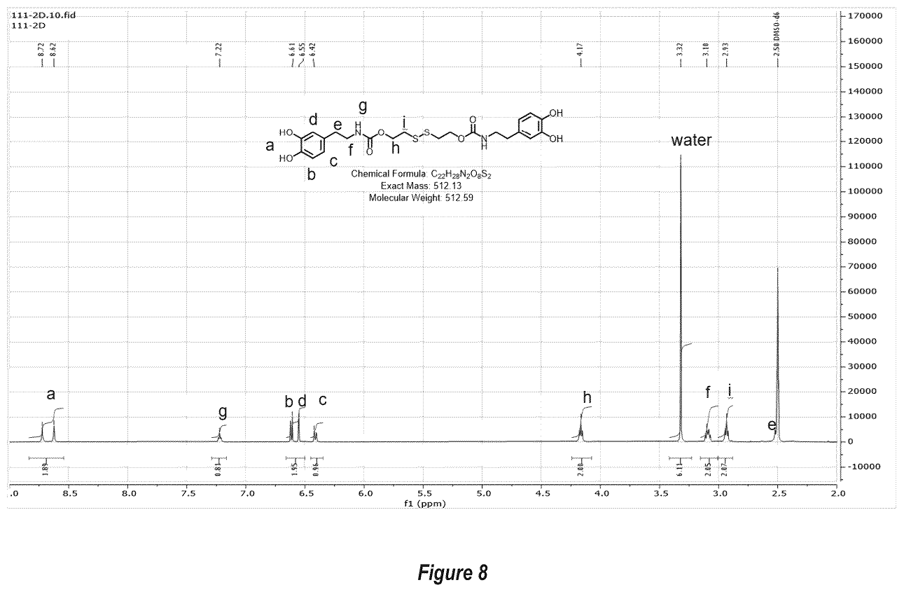

FIG. 8 shows the ¹H NMR spectrum of catechol 1 (400 MHZ, DMSO-d6).

FIG. 9 shows the HPLC (top) and mass spectrometry TIC (Total Ion Chromatogram) (bottom) traces for purified catechol 1.

FIG. 10 shows the mass spectrum (negative ion mode) of catechol 1.

FIG. 11 shows the ¹H NMR spectrum of tetraacid 2 (400 MHZ, DMSO-d6).

FIG. 12 shows the HPLC (top) and mass spectrometry TIC (Total Ion Chromatogram) (bottom) traces for purified tetraacid 2.

FIG. 13 shows the mass spectrum (negative ion mode) of tetraacid 2.

FIG. 14 shows data from a TdT extension assay, demonstrating the effect of chelators on TdT activity.

FIG. 15 shows a TdT extension assay using various chelator: metal ratios (e.g., 1:1, 2:1, 3:1, 4:1, 8:1, etc.). Not all chelators were tested at all concentrations. Omitted concentrations are denoted by a *

FIG. 16A shows a TdT extension assay using catechol 1 and tetraacid 2.

FIG. 16B shows a TdT extension assay using DTP at various concentrations.

FIG. 17 shows a TdT extension assay in the presence of a chelator (catechol 1), a reducing agent (TCEP), and catechol 1 pre-incubated with a reducing agent (TCEP). TCEP=tris(2-carboxyethyl) phosphine.

FIGS. 18A-18D show HPLC chromatograms of chelator solutions (FIG. 18A) DTP before bulk electrolysis, (FIG. 18B) 2-mercaptopyridine (degradation product) from commercial stock, (FIG. 18C) DTP solution after 24 hours without bulk electrochemical reduction, and (Figure D) DTP solution after 24 hours with bulk electrochemical reduction.

FIGS. 19A and 19B show HPLC-MS TIC of tetraacid 2 before (FIG. 19A) and after (FIG. 19B) bulk electrolysis.

### DETAILED DESCRIPTION

A description of example embodiments follows.

TABLE 1
Description of the Sequences

| | Parent | | | |
|---|---|---|---|---|
| | of the | | | |
| | En- | | | |
| | gineered | | | SEQ ID |
| Construct | Variant | Mutation | Sequence | NO |
| Wt | mTdT | wt | KISQYACQRRTTLNNYNQLFTDALDILAENDELRENEGSC | 1 |
| | | | LAFMRASSVLKSLPFPITSMKDTEGIPCLGDKVKSIIEGIIE | |
| | | | DGESSEAKAVLNDERYKSFKLFTSVFGVGLKTAEKWFR | |
| | | | MGFRTLSKIQSDKSLRFTQMQKAGFLYYEDLVSCVNRPE | |
| | | | AEAVSMLVKEAVVTFLPDALVTMTGGFRRGKMTGHDV | |
| | | | DFLITSPEATEDEEQQLLHKVTDFWKQQGLLLYCDILEST | |
| | | | FEKFKQPSRKVDALDHFQKCFLILKLDHGRVHSEKSGQQ | |
| | | | EGKGWKAIRVDLVMCPYDRRAFALLGWTGSRQFERDLR | |
| | | | RYATHERKMMLDNHALYDRTKRVFLEAESEEEIFAHLGL | |
| | | | DYIEPWERNA | |
| P1 | Pross1 | Kettner | KISQYAAQRRTTLNNYNKKFTDALDILAENAELRGNEGS | 2 |
| | | stabilized | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLHHQRVDSGKSG | |
| | | | QQEGKGWKAIRVDLVMAPYERRAFALLGWTGSRQFERD | |
| | | | LRRYARHERKMLLDNHALYDRTKNTFLRAESEEEIFAHL | |
| | | | GLEYIEPWERNA | |
| P2 | Pross2 | loop | KISQYAAQRRTTLNNYNKKFTDALDILAENAELRGNEGS | 3 |
| | | redesign | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| N1 | Pross2 | E32nc | KISQYAAQRRTTLNNYNKKFTDALDILAENAXLRGNEGS | 4 |
| | | (wherein | ALAFRRAASVLKSLPFPITSMKDTEGIPNLGDKVKRIIQEI | |
| | | X stands | LEDGESSEAKAVLNDERYQAFKLFTSVFGVGPKTAEKW | |
| | | for a non- | YRMGFRTLEDIRSDKTLRFTRMQKAGFLYYEDLVSPVTR | |
| | | canonical | PEAEAIEQLVKEAVWQFLPGATVTMTGGFRRGKKTGHD | |
| | | AA) | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| N2 | Pross2 | V152nc | KISQYAAQRRTTLNNYNKKFTDALDILAENAELRGNEGS | 5 |
| | | (wherein | ALAFRRAASVLKSLPFPITSMKDTEGIPNLGDKVKRIIQEI | |
| | | X stands | LEDGESSEAKAVLNDERYQAFKLFTSVFGVGPKTAEKW | |
| | | for a non- | YRMGFRTLEDIRSDKTLRFTRMQKAGFLYYEDLXSPVTR | |
| | | canonical | PEAEAIEQLVKEAVWQFLPGATVTMTGGFRRGKKTGHD | |
| | | AA) | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| N3 | Pross2 | P154nc | KISQYAAQRRTTLNNYNKKFTDALDILAENAELRGNEGS | 6 |
| | | (wherein | ALAFRRAASVLKSLPFPITSMKDTEGIPNLGDKVKRIIQEI | |
| | | X stands | LEDGESSEAKAVLNDERYQAFKLFTSVFGVGPKTAEKW | |
| | | for a non- | YRMGFRTLEDIRSDKTLRFTRMQKAGFLYYEDLVSXVTR | |
| | | canonical | PEAEAIEQLVKEAVWQFLPGATVTMTGGFRRGKKTGHD | |
| | | AA) | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C1 | Pross2 | E32C | KISQYAAQRRTTLNNYNKKFTDALDILAENACLRGNEGS | 7 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C7 | Pross1 | K102C | KISQYAAQRRTTLNNYNKKFTDALDILAENAELRGNEGS | 8 |
| | | | ALAFRRAASVLKSLPFPITSMKDTEGIPNLGDKVKRIIQEI | |
| | | | LEDGESSEAKAVLNDERYQAFCLFTSVFGVGPKTAEKWY | |
| | | | RMGFRTLEDIRSDKTLRFTRMQKAGFLYYEDLVSPVTRP | |
| | | | EAEAIEQLVKEAVWQFLPGATVTMTGGFRRGKKTGHDV | |
| | | | DFLITSPEATEEEEKQLLHKVTDWWKKQGLLLYEDIQEST | |
| | | | FEKFKLPSRKVDALDHFQKAFLILKLHHQRVDSGKSGQQ | |
| | | | EGKGWKAIRVDLVMAPYERRAFALLGWTGSRQFERDLR | |
| | | | RYARHERKMLLDNHALYDRTKNTFLRAESEEEIFAHLGL | |
| | | | EYIEPWERN | |
| C2 | Pross2 | V152C | KISQYAAQRRTTLNNYNKKFTDALDILAENAELRGNEGS | 9 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLCSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C3 | Pross2 | P154C | KISQYAAQRRTTLNNYNKKFTDALDILAENAELRGNEGS | 10 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSCVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C9 | Pross1 | E32C | KISQYAAQRRTTLNNYNKKFTDALDILAENACLRGNEGS | 11 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLHHQRVDSGKSG | |
| | | | QQEGKGWKAIRVDLVMAPYERRAFALLGWTGSRQFERD | |
| | | | LRRYARHERKMLLDNHALYDRTKNTFLRAESEEEIFAHL | |
| | | | GLEYIEPWERNA | |
| C4 | Pross1 | E29C | KISQYAAQRRTTLNNYNKKFTDALDILACNAELRGNEGS | 12 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLHHQRVDSGKSG | |
| | | | QQEGKGWKAIRVDLVMAPYERRAFALLGWTGSRQFERD | |
| | | | LRRYARHERKMLLDNHALYDRTKNTFLRAESEEEIFAHL | |
| | | | GLEYIEPWERNA | |
| C5 | Pross1 | E37C | KISQYAAQRRTTLNNYNKKFTDALDILAENAELRGNCGS | 13 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLHHQRVDSGKSG | |
| | | | QQEGKGWKAIRVDLVMAPYERRAFALLGWTGSRQFERD | |
| | | | LRRYARHERKMLLDNHALYDRTKNTFLRAESEEEIFAHL | |
| | | | GLEYIEPWERNA | |
| C7 | Pross1 | K102C | KISQYAAQRRTTLNNYNKKFTDALDILAENAELRGNEGS | 14 |
| | | | ALAFRRAASVLKSLPFPITSMKDTEGIPNLGDKVKRIIQEI | |
| | | | LEDGESSEAKAVLNDERYQAFCLFTSVFGVGPKTAEKWY | |
| | | | RMGFRTLEDIRSDKTLRFTRMQKAGFLYYEDLVSPVTRP | |
| | | | EAEAIEQLVKEAVWQFLPGATVTMTGGFRRGKKTGHDV | |
| | | | DFLITSPEATEEEEKQLLHKVTDWWKKQGLLLYEDIQEST | |
| | | | FEKFKLPSRKVDALDHFQKAFLILKLHHQRVDSGKSGQQ | |
| | | | EGKGWKAIRVDLVMAPYERRAFALLGWTGSRQFERDLR | |
| | | | RYARHERKMLLDNHALYDRTKNTFLRAESEEEIFAHLGL | |
| | | | EYIEPWERNA | |
| C8 | Pross2 | E29C | KISQYAAQRRTTLNNYNKKFTDALDILACNAELRGNEGS | 15 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C10 | Pross2 | E37C | KISQYAAQRRTTLNNYNKKFTDALDILAENAELRGNCGS | 16 |
| | | | ALAFRRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C11 | Pross2 | R44C | KISQYAAQRRTTLNNYNKKFTDALDILAENAELRGNEGS | 17 |
| | | | ALAFCRAASVLKSLPFPITSMKDTEGIPNLGDKVKRIIQEI | |
| | | | LEDGESSEAKAVLNDERYQAFKLFTSVFGVGPKTAEKW | |
| | | | YRMGFRTLEDIRSDKTLRFTRMQKAGFLYYEDLVSPVTR | |
| | | | PEAEAIEQLVKEAVWQFLPGATVTMTGGFRRGKKTGHD | |
| | | | VDFLITSPEATEEEEKQLLHKVTDWWKKQGLLLYEDIQE | |
| | | | STFEKFKLPSRKVDALDHFQKAFLILKLGPSPGKAIRVDL | |
| | | | VMAPYERRAFALLGWTGSRQFERDLRRYARHERKMLLD | |
| | | | NHALYDRTKNTFLRAESEEEIFAHLGLEYIEPWERNA | |
| C12 | Pross2 | K102C | KISQYAAQRRTTLNNYNKKFTDALDILAENAELRGNEGS | 18 |
| | | | ALAFRRAASVLKSLPFPITSMKDTEGIPNLGDKVKRIIQEI | |
| | | | LEDGESSEAKAVLNDERYQAFCLFTSVFGVGPKTAEKWY | |
| | | | RMGFRTLEDIRSDKTLRFTRMQKAGFLYYEDLVSPVTRP | |
| | | | EAEAIEQLVKEAVWQFLPGATVTMTGGFRRGKKTGHDV | |
| | | | DFLITSPEATEEEEKQLLHKVTDWWKKQGLLLYEDIQEST | |
| | | | FEKFKLPSRKVDALDHFQKAFLILKLGPSPGKAIRVDLVM | |
| | | | APYERRAFALLGWTGSRQFERDLRRYARHERKMLLDNH | |
| | | | ALYDRTKNTFLRAESEEEIFAHLGLEYIEPWERNA | |

### I. Definitions

Unless defined otherwise, all technical and scientific terms used herein have the same meaning as is commonly understood by one of skill in the art to which the claimed subject matter belongs. It is to be understood that the foregoing general description and the following detailed description are illustrative and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, “nucleotide molecule” refers to components of nucleic acids comprising a base, sugar, and phosphate moieties, both natural and unnatural, including nucleotides, ribonucleotides, and nucleotide analogs. As used herein, a nucleotide refers to a molecule comprising a nucleoside and one or more phosphate groups. As used herein, a nucleoside refers to a molecule comprising a nucleobase (e.g., adenine, thymine, cytosine, guanine, or uracil) and a five-carbon sugar (e.g., ribose or 2′-deoxyribose). Example natural nucleotides include, without limitation, ATP, UTP, CTP, GTP, ADP, UDP, CDP, GDP, AMP, UMP, CMP, GMP, dATP, dTTP, dCTP, dGTP, dADP, dTDP, dCDP, dGDP, dAMP, dTMP, dCMP, and dGMP. Example natural deoxyribonucleotides include dATP, dTTP, dCTP, dGTP, dADP, dTDP, dCDP, dGDP, dAMP, dTMP, dCMP, and dGMP. Example natural ribonucleotides include ATP, UTP, CTP, GTP, ADP, UDP, CDP, GDP, AMP, UMP, CMP, and GMP. For natural RNA, the uracil base is uridine. A nucleotide analog, or unnatural nucleotide, comprises a nucleotide which contains some type of modification to either the base, sugar, or phosphate moieties, such as, for example, a chemical modification. All chemical structures showing protonated triphosphates and/or sulfonates should be interpreted to include the protonated and the ionized salt forms in different buffers.

As used herein, “spacer” refers to a molecule that may be used to link two other molecules, although spacers may be present by themselves or attached to only one other molecule. A spacer may be an organic spacer (e.g., an aliphatic spacer, an alkyl spacer, an aromatic spacer, an alkylene glycol, a polyethylene glycol, a carbohydrate such as a sugar, and the like). In some embodiments, the spacer is a PEG spacer, a well-known inert spacer used in many biotechnological applications.

As used herein, a “tether” or “tether molecule” refers to the molecule covalently linking/connecting/attaching a TdT to a nucleotide molecule, optionally comprising a spacer.

The disclosure provides nucleic acid sequences and amino acid sequences having a certain degree of identity to a given nucleic acid sequence or amino acid sequence, respectively (a reference sequence). “Sequence identity” between first and second nucleic acid sequences indicates the percentage of nucleotides that are identical between the sequences; for example, if a first nucleic acid sequence is 95% identical to a second nucleic acid sequence, then the first nucleic acid sequence contains matches to 95% of the nucleotides in the second nucleic acid sequence. “Sequence identity” between two amino acid sequences indicates the percentage of amino acids that are identical between the sequences; for example, if a first amino acid sequence is 95% identical to a second amino acid sequence, then the first amino acid sequence contains matches to 95% of the nucleotides in the second amino acid sequence. The terms “% identical”, “% identity” or similar terms are intended to refer, in particular, to the percentage of nucleotides or amino acids which are determined to be identical using an optimal alignment between the sequences to be compared. Said percentage is purely statistical, and the differences between the two sequences may be but are not necessarily randomly distributed over the entire length of the sequences to be compared. Comparisons of two sequences are usually carried out by comparing said sequences after optimal alignment. The optimal alignment for a comparison may be carried out manually or with the aid of an appropriate algorithm such as the alignment algorithm by Needleman and Wunsch, 1970, J. Mol. Biol. 48, 443, or with the aid of computer programs using said algorithms (e.g., GAP, BESTFIT, and FASTA in Wisconsin Genetics Software Package, Genetics Computer Group, 575 Science Drive, Madison, Wis.).

As used herein, the terms “non-canonical amino acid” or “ncAA” or “non-canonical amino acid residue” or “nc” refer to an amino acid other than one of the 20 naturally occurring amino acids. Example non-canonical amino acids are described in Young et al., “Beyond the canonical 20 amino acids: expanding the genetic lexicon,” J. of Biological Chemistry 285 (15): 11039-11044 (2010), the disclosure of which is herein incorporated by reference.

As used herein, the terms “n” and “m” as they appear in chemical structures throughout the application refer to an integer from 1-12, unless defined otherwise.

The term “tetraacid 2” is used herein to refer to 3,14-bis(carboxymethyl)-4,13-dioxo-5,12-dioxa-8,9-dithia-3,14-diazahexadecanedioic acid.

### II. TdT Enzymes

The present disclosure addresses, among other things, the synthesis of a polymer wherein an enzyme (e.g., terminal deoxynucleotidyl transferase, referred to herein as TdT or Tdt) may mediate addition of monomers to a growing oligomer chain. Provided herein, for example, are methods for controlling the sequence in which the monomer units are added. The present disclosure addresses, among other things, de novo DNA synthesis with sequence control, using enzymatic methods. Current methods for DNA/RNA synthesis rely on classical phosphoramidite chemistry, but this chemistry is typically useful for sequences less than 200 base pairs in length and is prone to errors. Moreover, phosphoramidite chemistry requires use of toxic solvents and reagents.

Alternatively, use of enzymes to generate long sequences of DNA or RNA in a sequence-controlled manner could enable synthesis of long DNA/RNA sequences with limited error rates. De novo enzymatic DNA synthesis is relevant to many applications, including gene synthesis and biodefense (rapid bio-threat identification and countermeasure development). Enzymatic synthesis methods are performed in aqueous solutions, also providing an environmentally conscious solution for nucleic acid synthesis.

TdT enzymes are further described in Gouge, J. Mol. Biol. 425:4334-4352 (2013). Any TdT may be employed herein, including, but not limited to TdTs provided in SEQ ID Nos: 1-18, or TdTs comprising 90%, 91%, 92%, 93%, 94%, 95%, 96%, 97%, 98%, or 99% homology to any of the sequences in SEQ ID Nos: 1-18.

In some instances, the TdT molecule is attached to a nucleotide desired for addition to the nucleic acid being synthesized. In some embodiments, the TdT and the nucleotide are attached covalently by a linker that may be described as a tether and may comprise a spacer.

Additional arrangements of the TdT and nucleotide are described in International Application No. PCT/US2023/074179, the entire of the foregoing is incorporated herein by reference.

III Removal of the TdT from the Nucleic Acid Molecule

Because the TdT molecule is attached to the nucleotide, it becomes complexed with the nucleic acid molecule being synthesized after addition of the nucleotide. In some embodiments, TdT is deactivated or removed (e.g., in order to continue nucleic acid synthesis and generally to use the nucleic acid product).

In some embodiments, conditions sufficient to deactivate the TdT and remove it from the nucleic acid molecule comprise digestion by an enzyme; chemical deactivation; electrochemical deactivation; photochemical deactivation; or a combination thereof. In some embodiments, conditions sufficient to deactivate the TdT and remove it from the nucleic acid molecule comprise digestion by an enzyme.

In some embodiments, the enzyme is a protease or hydrolase. In some embodiments, the enzyme is a proteinase K. In some embodiments, the enzyme is a pH sensitive enzyme.

### a. pH Sensitive Enzymes Including Proteinase K

A number of pH sensitive enzymes may be used, including proteinase K. The pH sensitive enzyme may be a protease or a hydrolase. Any proteinase K that is pH sensitive may be used. Proteinase K, that is also Rnase and DNase free, may be obtained from GoldBio. The sequence of the GoldBio proteinase K and wildtype proteinase K is incorporated by reference herein. Other proteinase K variants may be used that comprising 90%, 91%, 92%, 93%, 94%, 95%, 96%, 97%, 98%, or 99% homology to the GoldBio and/or wildtype proteinase K.

A change in pH conditions may be caused by a change in voltage. A change in pH conditions may also be caused by addition of an acidic agent, basic agent, or a combination thereof to the buffer. A change in pH conditions may be caused by the addition of an agent chosen from glycine buffers (pH 1-2), sodium formate (pH 3-4), citrate phosphate (pH 3-8), phosphate buffers (pH 6-9), sodium acetate (pH 4.5-5.5) 2-(N-morpholino) ethanesulfonic acid (MES), pH 4.7-6), (4-(2-hydroxyethyl)-1-piperazineethanesulfonic acid) (HEPES) pH 6.8-8.2, tris(hydroxymethyl)aminomethane (Tris) (pH 7.1-9.1) and N-cyclohexyl-3-aminopropanesulfonic acid (CAPS) (pH 9.7-11.1).

A change in pH conditions is mediated by electrochemically reducing a pH mediator to convert it to a proton donor or electrochemically oxidizing a pH mediator to convert it to a proton sink. In some embodiments, the pH mediator is a quinone (e.g., 1,4-benzoquinone, 2,5-dihydroxy-1,4-benzoquinone, 2,5-dimethyl-1,4-benzoquinone, tetrafluoro-1,4-benzoquinone, anthraquinone-2,7-disulfonic acid (AQDS), 1,2-benzoquinone-3,5-disulfonic acid, 1,4-benzoquinone-3,5-disulfonic acid), ascorbate (e.g., dehydroascorbic acid), radical (e.g., 2,2,6,6-tetramethylpiperidine-1-oxyl (TEMPO), 4-hydroxy-2,2,6,6-tetramethylpiperidine-1-oxyl (4-OH-TEMPO), 4-[3-(trimethylammonio) propoxy]-2,2,6,6-tetramethylpiperidine-1-oxyl (TMAP-TEMPO)), flavin (e.g., riboflavin, flavin mononucleotide (FMN), flavin adenine dinucleotide (FAD)), phenazine (e.g., disodium 3,3′-(phenazine-2,3-diylbis(oxy))bis(propane-1-sulfonate) (DSPZ), 7,8-dihydroxyphenazine-2-sulfonic acid (DHPS), 2,2′-(phenazine-1,8-diyl)bis(ethane-1-sulfonate) (1,8-ESP)), phenothiazine (e.g., methylene blue, methylene green, Azure A, Azure B, Azure C, thionine), polyoxometalate (e.g., silicotungstic acid, phosphotungstic acid), alloxazine (e.g., alloxazine, 7,8-hydroxyalloxazine, 7,8-dimethoxyalloxazine, Alloxazine-7,8-carboxylic acid, 7,8-dimethylalloxazine), or a combination thereof. In some instances, the pH mediator is benzophenone. In some embodiments, the pH mediator undergoes proton-coupled electron transfer (PCET) redox reactions in pH range of interest. In some embodiments, the reduction potential of the pH mediator is within solvent stability window. In some embodiments, the reduction potential of the pH mediator is sufficiently reducing to avoid oxidative damage to DNA (i.e., oxidation of guanine to 8-oxo-2′-deoxyguanosine at 1.29 V vs. NHE). In some embodiments, the pH mediator is chemically stable in both protonated and deprotonated states over a wide range of pH conditions (e.g., acidic, neutral, basic). In some embodiments, the pH mediator is soluble in both protonated and deprotonated state over a wide range of pH conditions (e.g., acidic, neutral, basic).

In some embodiments, the pH sensitive enzyme (such as proteinase K) inactivation step comprises lowering the pH below 5 or raising the pH above 10. In some embodiments, the pH sensitive enzyme (such as proteinase K) inactivation step comprises lowering the pH into a range from 3.5 to 5. In some embodiments, the pH sensitive enzyme (for example, proteinase K) inactivation step comprises increasing the pH into a range from 10 to 12. In some embodiments, the pH sensitive enzyme (such as proteinase K) activation step comprises changing (e.g., increasing, lowering) the pH into a range from 5 to 10. In some embodiments, the pH sensitive enzyme (such as proteinase K) activation step comprises changing the pH into a range from 6 to 8. In some embodiments, the pH sensitive enzyme (such as proteinase K) activation step comprises changing the pH into a range from 6.5 to 7.5. In some embodiments, the pH sensitive enzyme (such as proteinase K) activation step comprises changing the pH into a range from 7.0 to 7.5.

Proteinase K and its control through the pH of the reaction solution are further described in Yazawa, ACS Catal. 6:3036-3046 (2016); Schatz, Nature Portfolio Scientific Reports 12:8274 (9 pages) (2022); Yang, Protein Expression and Purification 122:38-44 (2016); Kadambar, Chem. Commun. 56:13800 (4 pages) (2020); O'Sullivan, Electrochimica Acta 395:139113 (9 pages) (2021).

Electrochemical pH change as it applies to multiple functions in this invention may be further described in WO 2022/204301 (Lackey) and Pande, J. Phys. Chem. Lett. 11:7042-7048 (2020).

### IV. Cleavable Chelating Agents

A cleavable chelating agent that is able to (1) sequester a metal cofactor and (2) release it upon cleavage may be used. A chelating agent may be cleavable by a reducing agent, an oxidizing agent, or a redox-mediating agent.

In some embodiments, the reducing agent comprises dithiothreiotol (DTT), leucomethylene blue, sodium dithionite, sodium bisulfite, tris(2-carboxyethyl) phosphine (TCEP), sodium thiosulfate, or a combination thereof. In some embodiments, the reducing agent comprises tris(2-carboxyethyl) phosphine. In some embodiments, the oxidizing agent comprises sodium periodate, 2,3-dichloro-5,6-dicyanobenzoquinone (DDQ), hydrogen peroxide, or a combination thereof. In some embodiments, the redox-mediating agent comprises Methylene Blue (MB), Methyl Viologen (MV), bis-(trimethylammonio) propyl viologen (BTMAP-Vi), Riboflavin (RF), Flavin mononucleotide (FMN), Flavin adenine dinucleotide (FAD), 2,5-dihydroxy-1,4-benzoquinone (DHBQ), 9,10-anthraquinone-2,7-disulfonic acid (AQDS), Hexaamineruthenium (III) chloride (RuHex), or a combination thereof.

In some embodiments, cleaving the chelating agent occurs through a voltage change at an electrode. In some embodiments, the voltage change causes reduction or oxidation at the electrode. In some embodiments, the voltage potential range is about 0 V to about 1.2 V versus reversible hydrogen electrode (RHE). In some embodiments, the voltage potential for cleavage of the chelating agent is from about +1.05V vs. SHE to about −0.18V vs. SHE at 25° C. and pH 3; about +0.84 V vs. SHE to about −0.38 V vs. SHE at 25° C. and pH 6.5; or about +0.64V vs. SHE to about −0.59V vs. SHE at 25° C. and pH 10. Further information on voltage change and use of electrodes may be found at Materialsproject_org. Further information may be found in Jain, Apl Materials 1:011002 (12 pages) (2013).

The chelating agent may be added to a reaction solution in a complex with a metal cofactor. In some embodiments, a metal cofactor comprises iron, magnesium, manganese, cobalt, copper, zinc, molybdenum, or a combination thereof.

The chelating agent may comprise a cleavage site comprising a disulfide bond.

The chelating agent may comprise a catechol group as shown as follows:

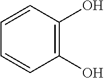

In some embodiments, a chelating agent comprises a tetracarboxylic acid group. A chelating agent may comprise an EDTA analog, which includes any chelating agent that comprises a tetracarboxylic acid group. Tetracarboxylic acids are a class of organic compounds that contain four carboxyl (COOH) groups. In some embodiments, the four carboxylic acid groups are in close enough proximity to all bind to and chelate a metal. In some embodiments, one, two, three, or four carboxylic acid groups are acetic acid groups. EDTA is shown as follows:

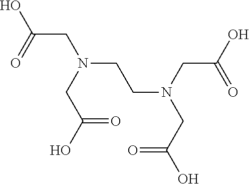 [SMILES: O=C(O)CN(CCN(CC(=O)O)CC(=O)O)CC(=O)O]

### Ethylenediaminetetraacetic Acid

In some embodiments, the chelating agent comprises the compound of formula 1 or formula 2, shown as follows:

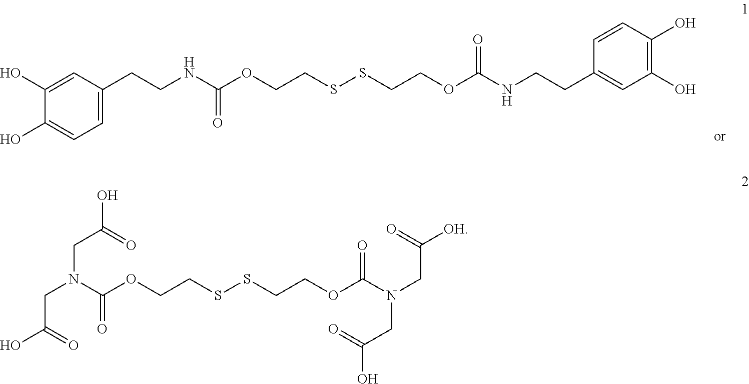 [SMILES: O=C(NCCc1ccc(O)c(O)c1)OCCSSCCOC(=O)NCCc1ccc(O)c(O)c1 ; O=C(O)CN(CC(=O)O)C(=O)OCCSSCCOC(=O)N(CC(=O)O)CC(=O)O]

### V. Methods of Nucleic Acid Synthesis

a. Types of Nucleic Acid Synthesis

Methods described herein apply to multiple types of nucleic acid synthesis, for example, DNA synthesis.

In some instances, a method of nucleic acid molecule synthesis occurs on a solid surface having multiple sites for nucleic acid molecule synthesis. In some embodiments, multiple sites on a solid surface are each functionally coupled to an electrode capable of a localized voltage change. In some embodiments, a localized voltage change creates a localized change in pH conditions. In some embodiments, each localized voltage change activates the TdT at only a single site on a solid surface.

b. A First Method of Nucleic Acid Synthesis

In some embodiments, a first method of nucleic acid synthesis controls the activity of the TdT by depriving it of metal cofactors needed for its activity. Cleavable chelating agents may be used to sequester metal cofactors until the desired time in a reaction or the desired location in a multiplexed reaction. Upon cleavage of the chelating agent, metal cofactors are released into the reaction solution and the reaction can proceed.

In some embodiments, a first method of nucleic acid molecule synthesis comprises one or more steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker;
(c) providing at least one metal cofactor complexed to at least one cleavable chelating agent, wherein the chelating agent is capable of releasing the metal cofactor upon cleavage;
(d) selectively activating the TdT by cleaving the chelating agent;
(e) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand;
(f) exposing the TdT to conditions sufficient to deactivate it and remove it from the nucleic acid molecule; and
(g) repeating steps (b) through (f) thereby synthesizing a nucleic acid molecule.

In some embodiments, a first method of nucleic acid molecule synthesis comprises the steps of:
(h) providing a single-stranded nucleic acid molecule comprising a 5′ and a 3′ end;
(i) providing an engineered terminal deoxynucleotidyl transferase (TdT), wherein the engineered TdT comprises a nucleotide molecule covalently attached to the TdT via a cleavable linker;
(j) providing at least one metal cofactor complexed to at least one cleavable chelating agent, wherein the chelating agent is capable of releasing the metal cofactor upon cleavage;
(k) selectively activating the TdT by cleaving the chelating agent;
(l) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand;
(m) exposing the TdT to conditions sufficient to deactivate it and remove it from the nucleic acid molecule; and
(n) repeating steps (b) through (f) thereby synthesizing a nucleic acid molecule.
   c. A Second Method of Nucleic Acid Synthesis

In some embodiments, a second method of nucleic acid synthesis controls the activity of the TdT by deactivating it through a pH sensitive enzyme that is added to the reaction solution and activated at the desired time in the reaction and/or at the desired location in a multiplexed reaction. In some embodiments, upon activation of the pH sensitive enzyme, the TdT is destroyed and removed from the nucleic acid synthesis reaction. Other benefits include a generalized cleaning and removal of proteinaceous contaminants in the reaction solution or on the surface of a reaction site.

In some embodiments, a second method of nucleic acid molecule synthesis in a buffer comprises one or more steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker and at least one metal cofactor;
(c) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(d) providing an inactivated pH sensitive enzyme, wherein the pH sensitive enzyme is inactivated by pH conditions of the buffer;
(e) activating the pH sensitive enzyme through a change in pH conditions in the buffer and digesting the TdT releasing the extended nucleic acid strand;
(f) inactivating the pH sensitive enzyme through a change in pH conditions; and
(g) repeating steps (c) through (f) at least once thereby synthesizing a nucleic acid molecule.

In some embodiments, a second method of nucleic acid molecule synthesis in a buffer comprises the steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker and at least one metal cofactor;
(c) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(d) providing an inactivated pH sensitive enzyme, wherein the pH sensitive enzyme is inactivated by pH conditions of the buffer;
(e) activating the pH sensitive enzyme through a change in pH conditions in the buffer and digesting the TdT releasing the extended nucleic acid strand;
(f) inactivating the pH sensitive enzyme through a change in pH conditions; and
(g) repeating steps (c) through (f) at least once thereby synthesizing a nucleic acid molecule.
   d. A Combined Method of Nucleic Acid Synthesis

The two methods may also be combined for further improved benefits.

In some embodiments, a third method of nucleic acid molecule synthesis in a buffer comprises one or more steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker;
(c) providing at least one metal cofactor complexed to at least one cleavable chelating agent, wherein the chelating agent is capable of releasing the metal cofactor upon cleavage;
(d) selectively activating the TdT by cleaving the chelating agent;
(e) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(f) providing an inactivated pH sensitive enzyme, wherein the pH sensitive enzyme is inactivated by pH conditions of the buffer;
(g) activating the pH sensitive enzyme through a change in pH conditions in the buffer and digesting the TdT releasing the extended nucleic acid strand;
(h) inactivating the pH sensitive enzyme through a change in pH conditions; and
(i) repeating steps (c) through (h) at least once thereby synthesizing a nucleic acid molecule.

In some embodiments, a third method of nucleic acid molecule synthesis in a buffer comprises the steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker;
(c) providing at least one metal cofactor complexed to at least one cleavable chelating agent, wherein the chelating agent is capable of releasing the metal cofactor upon cleavage;
(d) selectively activating the TdT by cleaving the chelating agent;
(e) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(f) providing an inactivated pH sensitive enzyme, wherein the pH sensitive enzyme is inactivated by pH conditions of the buffer;
(g) activating the pH sensitive enzyme through a change in pH conditions in the buffer and digesting the TdT releasing the extended nucleic acid strand;
(h) inactivating the pH sensitive enzyme through a change in pH conditions; and
(i) repeating steps (c) through (h) at least once thereby synthesizing a nucleic acid molecule.

### VI. Systems for Enzymatic Nucleic Acid Synthesis

Systems for enzymatic DNA systems are described herein. In some embodiments, the system for enzymatic DNA synthesis comprises components described in Sections II (TdT Enzymes) and III (Removal of the TdT from the Nucleic Acid Molecule).

In some embodiments, the system for enzymatic DNA synthesis comprises: an engineered TdT described herein; and two or more electrodes on a surface. In some embodiments, the system for enzymatic DNA synthesis comprises: an engineered TdT described herein; and two or more electrodes on a surface, along with other components described herein. In some embodiments, the electrodes enable an applied potential or galvanic field to be supplied locally to the device or system thus enabling generation of active redox shuttle via reduction or oxidation at one or more electrodes) above, along with two or more electrodes on a surface. In some embodiments, the electrodes enable an applied potential or galvanic field to be supplied locally to the device or system thus enabling reduction or oxidation at one or more electrodes. In some embodiments, the electrodes enable an applied potential or galvanic field to be supplied locally to the device or system thus enabling consumption or generation of protons via the reduction or oxidation of a soluble pH mediator at one or more electrodes. In some embodiments, the electrode where the soluble pH mediator or redox shuttle is generated is comprised of a smaller surface area than the other electrode to which it is electrically coupled. In some embodiments, if a cathodic process is used, then the cathode may be comprised of less active surface area than the anode by (a) having less electroactive area, or (b) being coupled to multiple anodes which in combination provide substantially more area than the cathode. In some embodiments, the surface, or electroactive, or area aspect ratios for the counter electrode relative to the working electrode, or C/E aspect ratio is at least, for example, 3:1, 6:1, 10:1, 100:1, 1000:1, or higher. In some embodiments, all the electrodes lie in a single plane. In some embodiments, a combination of electrodes may lie in multiple planes. In some embodiments, the electrodes are oriented parallel to perpendicular with respect to one another during the current path. In some embodiments, the system has an inter-electrode gap (i.e., distance between electrodes included in the current path) of ≤100 μm, ≤10 μm, ≤1 μm, ≤100 μm, ≤10 nm, ≤1 nm, ≤100 pm, ≤10 pm, or ≤1 pm.

In some embodiments, the nucleic acid synthesis occurs in a buffer comprising a buffering agent, a supporting electrolyte, a hydrotropic agent, or a combination thereof. In some embodiments, the buffering agent is sodium citrate, potassium hydrogen phosphate, or potassium dihydrogen phosphate. In some embodiments, the supporting electrolyte is sodium chloride, sodium salts, lithium salts, potassium salts, or magnesium salts. In some embodiments, the hydrotropic agent is caffeine, urea, nicotinamide (NA), or a combination thereof. In some embodiments, the buffer comprises methylene blue, citrate-phosphate buffer, and sodium chloride. In some embodiments, the buffer comprises methylene blue, citrate-phosphate buffer, sodium chloride, and nicotinamide. In some embodiments, the buffer comprises flavin mononucleotide, citrate-phosphate buffer, sodium chloride, and nicotinamide. In some embodiments, the buffer comprises 10 mM methylene blue, 25 mM citrate-phosphate buffer at pH 3.4, and 100 mM sodium chloride. In some embodiments, the buffer comprises 10 mM methylene blue, 25 mM citrate-phosphate buffer at pH 3.4, 100 mM sodium chloride, and 1 M nicotinamide. In some embodiments, the buffer comprises 50 mM flavin mononucleotide, 25 mM citrate-phosphate buffer at pH 3.4, 1 M sodium chloride, and 1 M nicotinamide.

In some embodiments disclosed herein are systems for nucleic acid synthesis, comprising: an engineered TdT, wherein the engineered TdT comprises a nucleotide molecule covalently attached to the TdT via a cleavable linker; at least one metal cofactor complexed to at least one cleavable chelating agent; a redox shuttle solution; and two or more electrodes on a surface.

In other embodiments disclosed herein are systems for nucleic acid synthesis, comprising: an engineered TdT, wherein the engineered TdT comprises a nucleotide molecule covalently attached to the TdT via a cleavable linker and at least one metal cofactor; an inactivated pH sensitive enzyme; a redox shuttle solution; and two or more electrodes on a surface.

In yet other embodiments disclosed herein are systems for nucleic acid synthesis, comprising: an engineered TdT, wherein the engineered TdT comprises a nucleotide molecule covalently attached to the TdT via a cleavable linker; an inactivated pH sensitive enzyme; at least one metal cofactor complexed to at least one cleavable chelating agent; a redox shuttle solution; and two or more electrodes on a surface.

In some embodiments, the pH sensitive enzyme is a protease or hydrolase. In some embodiments, the pH sensitive enzyme is proteinase K.

In some embodiments, the chelating agent comprises a cleavage site comprising a disulfide bond. In some embodiments, the chelating agent comprises a catechol group. In other embodiments, the chelating agent comprises an EDTA analog. In some embodiments, the chelating agent comprises a tetracarboxylic acid group.

In some embodiments, the chelating agent comprises the compound of formula 1 or formula 2, shown as follows:

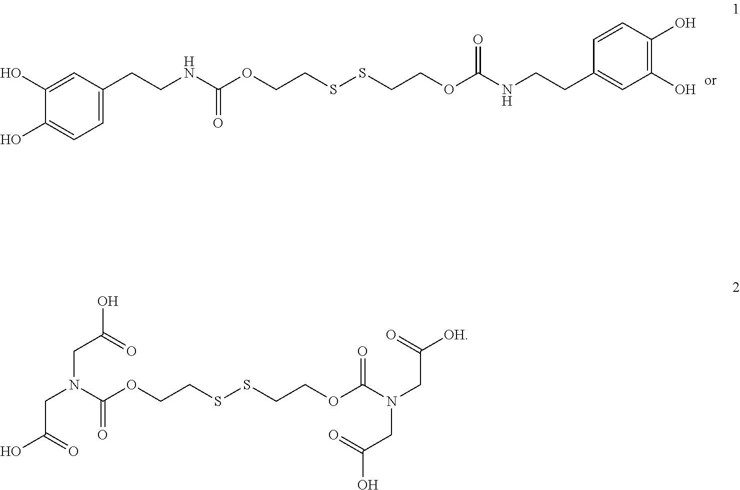 [SMILES: O=C(NCCc1ccc(O)c(O)c1)OCCSSCCOC(=O)NCCc1ccc(O)c(O)c1 ; O=C(O)CN(CC(=O)O)C(=O)OCCSSCCOC(=O)N(CC(=O)O)CC(=O)O]

In some embodiments, the at least one metal cofactor comprises iron, magnesium, manganese, cobalt, copper, zinc, molybdenum, or a combination thereof.

In some embodiments, the two or more electrodes comprise a counter electrode and a working electrode, and a surface area ratio between the counter electrode and the working electrode is at least about 3:1 (e.g., at least about 6:1, 10:1, 100:1, 1000:1, or higher, from about 3:1 to about 100:1, from about 3:1 to about 10:1, etc.).

In some embodiments, a plurality of the electrodes lie in a single plane or multiple planes. In some embodiments, a plurality of the electrodes are oriented parallel to perpendicular with respect to one another during a current path.

In some embodiments, the system has an interelectrode gap of less than about 100 μm (e.g., about 1 nm, about 10 nm, about 100 nm, about 1 μm, about 10 μm, about 100 μm).

In some embodiments, the system has an interelectrode gap of less than about 10 nm (e.g., from about 0.1 μm to about 5 nm).

In some embodiments, the redox shuttle solution comprises at least one soluble redox shuttle in combination with a buffer, a supporting electrolyte, a hydrotropic agent, or a combination thereof.

In some embodiments, the buffer comprises sodium citrate, potassium hydrogen phosphate, potassium dihydrogen phosphate, or a combination thereof.

In some embodiments, the supporting electrolyte comprises a sodium salt, lithium salt, potassium salt, magnesium salt, or a combination thereof.

In some embodiments, the hydrotropic agent comprises caffeine, urea, nicotinamide, or a combination thereof.

In some embodiments, the redox shuttle solution comprises methylene blue, flavin mononucleotide, citrate-phosphate buffer, sodium chloride, nicotinamide, or a combination thereof.

All of the references cited in the specification, including the Examples, are incorporated by reference for all of their teachings relevant to the present disclosure.

### EXAMPLES

### Example 1. Electrochemical Control of Proteinase K Via pH

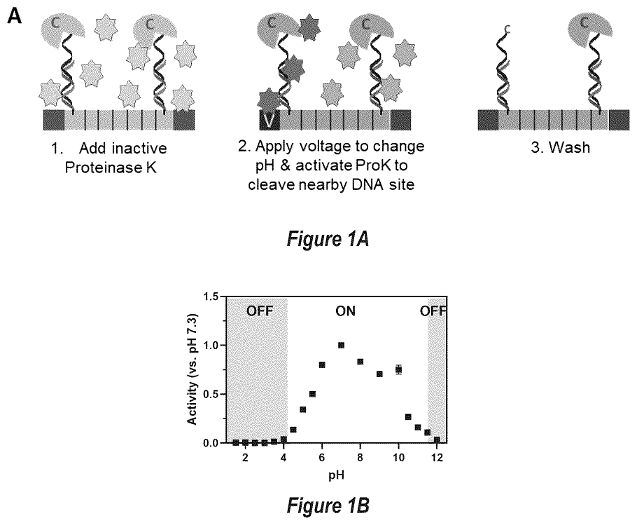

Another method of deblocking after nucleotide addition using a conjugated enzyme is to digest the protein around the extended DNA. Proteinase K (ProK) is a hydrolase that digests proteins at aliphatic and aromatic residues making it a broad-spectrum protease¹ that could be used to fully digest TdT releasing the extended DNA for the next round of addition. This enzymatic cleavage step could potentially be spatially controlled via an electrochemically driven pH change, as depicted in FIG. 1A. ProK would be introduced in a pH in which it is inactive. A voltage would then be applied to electrodes near the DNA site to be cleaved for the next addition. This would cause a shift in the pH near this active electrode that would re-activate ProK to digest nearby TdT molecules, deblocking the DNA for subsequent nucleotide addition. ProK has some natural pH sensitivity¹⁻² and could be further engineered to shift the activity profile into a desirable pH range. Preliminary data indicates that ProK has a complete dropoff in activity below pH 5 and above pH 10 (FIG. 1B). Additionally, activity can be recovered after PH neutralization (FIG. 1C). As an example of a base-OFF/neutral-ON pH switch, there is negligible activity at pH 12.5, while activity increases to between 0.5 and 1 after pH neutralization. A literature search indicates that depending on the conditions and electrode geometry we can expect to produce a pH shift anywhere from <1 unit up to several units³⁻⁵, and various buffers, voltages, and current densities can be explored to achieve the desired range.

Various pH mediators may be used to control the local pH for ProK activation or deactivation. For example, a buffer system (e.g., phosphate, citrate, tris buffer), solvent (e.g., water, methanol, acetonitrile, dimethyl sulfoxide), secondary pH mediator (e.g., small molecules or transition metal complexes that participate in proton transfer reactions such as quinones and phenols) may be employed to shift local pH via electrochemical activation.

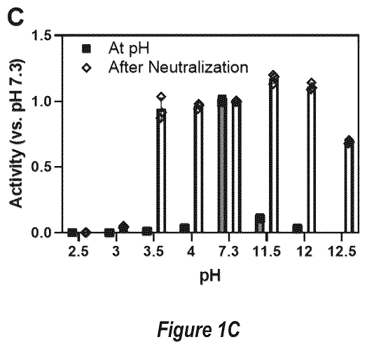

FIG. 2 illustrates an example method of inducing a pH change by applying localized voltage in the presence of a proton donor/acceptor, R. At the anode (positive electrode), the protonated pH mediator HₓR is oxidized, generating R, xH⁺, and xe⁻ where is x is the number of protons and electrons involved in the redox reaction. This generates a local decrease in pH around the electrode. Similarly, at the cathode (negative electrode), the deprotonated pH mediator R is reduced and protonated to yield H₂R. Depending on the pKₐ value(s) of oxidized/reduced R, the local pH, and the voltage, the number of protons and electrons may differ.

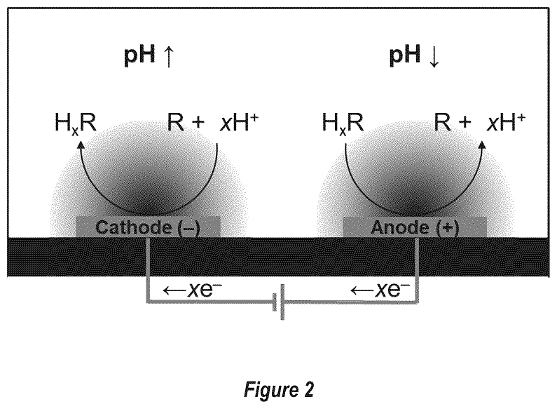

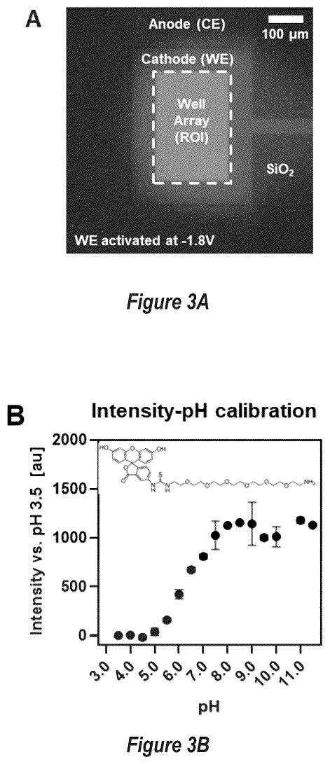

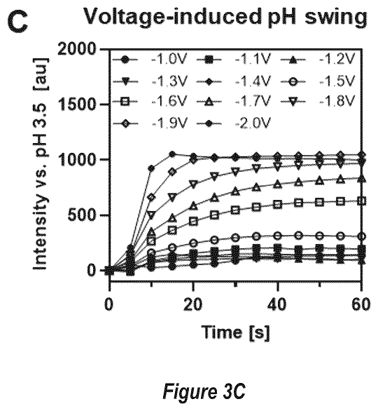

FIGS. 3A-C show how voltage-induced pH change can be visualized in real-time with a freely diffusing non-electroactive fluorescent probe. FIG. 3A shows a fluorescent microscope image of a microfabricated set of planar electrodes in the presence of an aqueous electrolyte containing 100 mM NaCl, 25 mM buffer, 0.5 mM 1,4-benzoquinone, 0.5 mM 2,5-dihydroxy-1,4-benzoquinone, and 1 mM fluorescein-PEG6-amine (FLPA, structure shown in inset). A large Pt counter electrode (CE, anode) surrounds a smaller Pt working electrode (WE, cathode). The two electrodes are separated by a 10 μm dielectric SiO₂ region. The WE (410 μm×510 μm) contains a microarray of 300-nm diameter wells that cannot be optically resolved but appear as a smaller square region (˜250 μm×325 μm), made brighter by optical interference. The microarray is used as the region of interest (ROI) for all subsequent analysis. Images were acquired with an Excelitas X-Cite Xylis XT720S (360-720 nm LED) source, 1s exposure time, and GFP (525/50 nm) filter set.

FIG. 3B shows the average pixel intensity in the ROI as a function of bulk electrolyte pH, baselined (i.e. zeroed) to values at pH 3.5. Data points represent the average of three technical replicates, and error bars represent one standard deviation. The intensity follows a sigmoidal relationship as the fraction of deprotonated, fluorescent FLPA (inset) increases with pH (pKₐ˜6.5). Thus, FLPA is sensitive to an acid-OFF/neutral-ON pH switch over pH 5 to 8. The visualization strategy is amenable to other non-electroactive probes with different pKₐ values.

FIG. 3C shows the change in intensity as a function of time with different applied voltages (i.e., constant voltage at −1.0V to −2.V for up to 1 minute) to the working electrode, demonstrating a tunable pH swing across the dynamic range of the FLPA intensity-pH calibration curve.

### References for Example 1

(1) H. Yang, et al., High-level expression of Proteinase K from Tritirachium album Limber in Pichia pastoris using multi-copy expression strains, Protein Expr Purif 122, p. 38-44, 2016 (10.1016/j.pep.2016.02.006).
(2) K. Yazawa, et al., Derivatization of Proteinase K with Heavy Atoms Enhances Its Thermal Stability, ACS Catal 6, p. 3036-3046, 2016 (10.1021/acscatal.6b00100).
(3) M. Schatz, et al., Quantifying local pH changes in carbonate electrolyte during copper-catalysed CO2 electroreduction using in operando 13C NMR, Sci. Rep. 12, p. 8274, 2022 (10.1038/s41598-022-12264-8).
(4) V. K. Kadambar, et al., Electrochemical control of the catalytic activity of immobilized enzymes, Chem. Commun. 56, p. 13800, 2020 (10.1039/d0cc06190e).
(5) B. O'Sullican, et al., A simulation and experimental study of electrochemical pH control at gold interdigitated electrode arrays, Electrochimica Acta 395, p. 139113, 2021 (10.1016/j.electacta.2021.139113).
(6) H. S. Jung, et al., CMOS electrochemical pH localizer-imager. Science advances, 8 (30), eabm6815, 2022 (10.1126/sciadv.abm6815).

### Example 2. Enzymatic Control Through Redox-Mediated Chelators

Enzymes often rely on metal cofactors to maintain enzymatic activity. A common mechanism for either inhibiting enzymatic activity or quenching/stopping enzymatic reactions is addition of a chelating agent to sequester the metal cofactors. Chelating agents, or chelators, are molecules that coordinate and sequester metal cations, making the metal cations unavailable for interaction with enyzmes or other solution components. For TdT to be active, metal cofactors, including iron, magnesium, manganese, cobalt, copper, zinc, molybdenum, or a combination thereof, must be present. TdT activity can be inhibited or quenched by addition of chelating agents, typically EDTA (ethylenediamine tetraacetic acid).

Examples of controlling enzyme activity, in particular, TdT activity through use of chelating agents have been disclosed. Use of a photocleavable chelator for controlling TdT activity and subsequent photocontrol of ssDNA synthesis was described in Lee et al. 10.1101/2020.02.19.956888. In this publication, photocaged EDTA analogs bound to cobalt were incubated with nucleotide triphosphates, TdT, and surface-immobilized ssDNA. Upon irradiation with UV light, bonds in photocaged EDTA chelator were cleaved, lowering affinity for Co and releasing Co into solution to interact with TdT and actuate TdT activity. Many examples of photolabile chelators have been disclosed, for example Cui et al. “New Photolabile BAPTA-Based Ca2+ Cages with Improved Photorelease,” J. Am. Chem. Soc. 2012, 134, 7733-7740 and Ellis-Davies. “Neurobiology with Caged Calcium,” Chem. Rev. 2008, 108,1603-1613.

We are disclosing an analogous method for controlling TdT activity wherein the chelators are designed to have a redox-cleavable site. In this case, the chelators would sequester metal cofactors until actuation by a reducing or oxidizing agent (or redox mediating agent). These chelator designs contain a redox-cleavable moiety (a disulfide) and chelating functionalities, either a tetraacetic acid function (EDTA analog) or use of catechols as chelating agents. Representative redox-cleavable chelators and synthetic protocols are disclosed below in Example 3.

### Example 3. Synthesis of Cleavable Metal Chelators

### A. General Structures

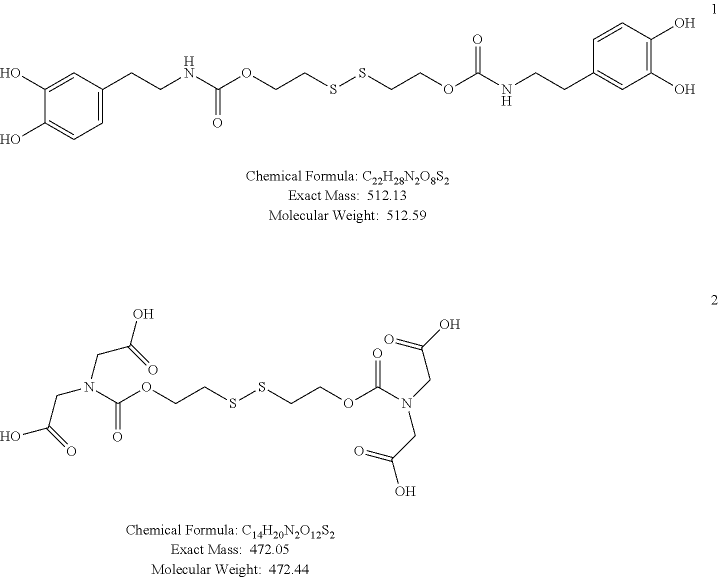 [SMILES: O=C(NCCc1ccc(O)c(O)c1)OCCSSCCOC(=O)NCCc1ccc(O)c(O)c1 ; O=C(O)CN(CC(=O)O)C(=O)OCCSSCCOC(=O)N(CC(=O)O)CC(=O)O]

### B. Methods and Materials

All materials were purchased from Sigma-Aldrich and used as-received unless otherwise mentioned. Dopamine hydrochloride was purchased from ChemImpex.

### C. Synthesis of Products

### Synthesis of 3,14-bis(carboxymethyl)-4,13-dioxo-5,12-dioxa-8,9-dithia-3,14-diazahexadecanedioic acid

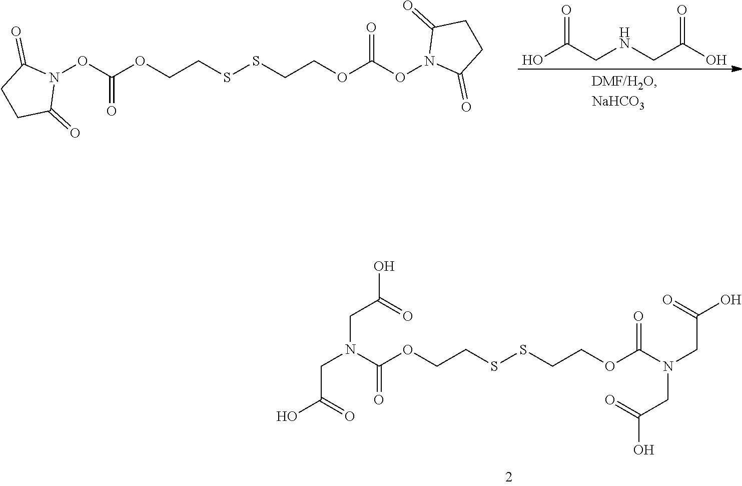 [SMILES: O=C(OCCSSCCOC(=O)ON1C(=O)CCC1=O)ON1C(=O)CCC1=O ; O=C(O)CNCC(=O)O ; O=C(O)CN(CC(=O)O)C(=O)OCCSSCCOC(=O)N(CC(=O)O)CC(=O)O]

In a 5 mL vial, 50 mg (0.115 mmol) of bis(2,5-dioxopyrrolidin-1-yl) (disulfanediylbis(ethane-2,1-diyl)) decarbonate was dissolved into 1 mL of N,N′-dimethyl formamide (DMF). To this solution, 60 mg (0.451 mmol, 3.9 eq.) of iminodiacetic acid was added and mixed until fully dissolved. The solution was diluted to 2 mL with DMF and 250 mg of sodium bicarbonate was added (2.97 mmol, 25.8 eq). The reaction was sealed and allowed to stir for 16 hours. Conversion was tested by LC-MS before purification, if incomplete, extra iminodiacetic acid and sodium bicarbonate was added before stirring for additional time.

Once conversion was confirmed, The sample was acidified to pH ˜2 with 2N HCl. The acidified reaction solution was then purified via a biotage with gradient of acetonitrile/water mixture. The solvent from the desired fraction was removed by lyophilization before testing (10 mg, 18% yield).

### Synthesis of disulfanediylbis(ethane-2,1-diyl) bis((3,4-dihydroxyphenethyl)carbamate)

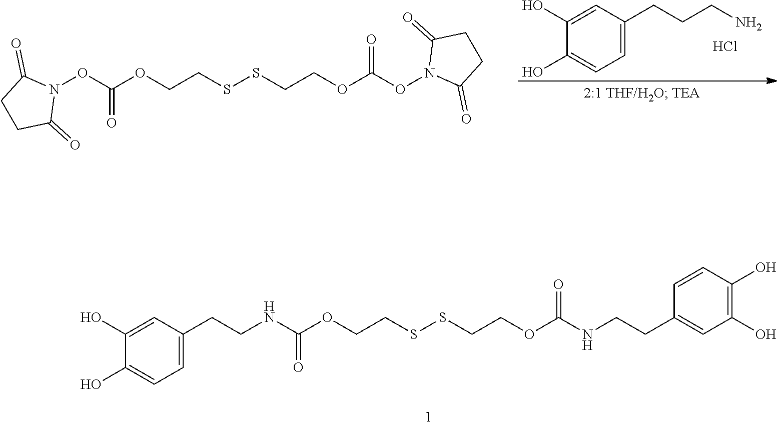 [SMILES: O=C(OCCSSCCOC(=O)ON1C(=O)CCC1=O)ON1C(=O)CCC1=O ; NCCCc1ccc(O)c(O)c1 ; Cl ; O=C(NCCc1ccc(O)c(O)c1)OCCSSCCOC(=O)NCCc1ccc(O)c(O)c1]

In a 5 mL vial, 50 mg (0.115 mmol) of Bis(2,5-dioxopyrrolidin-1-yl) (disulfanediylbis(ethane-2,1-diyl)) decarbonate was dissolved into 1 mL of THF and water (50% v/v). To this solution, 80 mg (0.421 mmol, 3.6 eq.) of dopamine hydrochloride was added and mixed until full dissolved. The solution was diluted to 2 mL with 50:50 THF/water (% v/v) and 250 mg of sodium bicarbonate was added (3.125 mmol, 26.5 eq). The reaction was sealed and allowed to stir for 16 hours. Conversion was tested by LC-MS before purification, if incomplete, extra dopamine hydrochloride and sodium bicarbonate was added before stirring for additional time.

Once conversion was confirmed, the sample was acidified to PH ˜2 with 2N HCl. The acidified reaction solution was then purified via a biotage with gradient of acetonitrile/water mixture. The solvent from the desired fraction was removed by lyophilization before testing (20 mg, 33.9% yield).

### D. Analytical Information

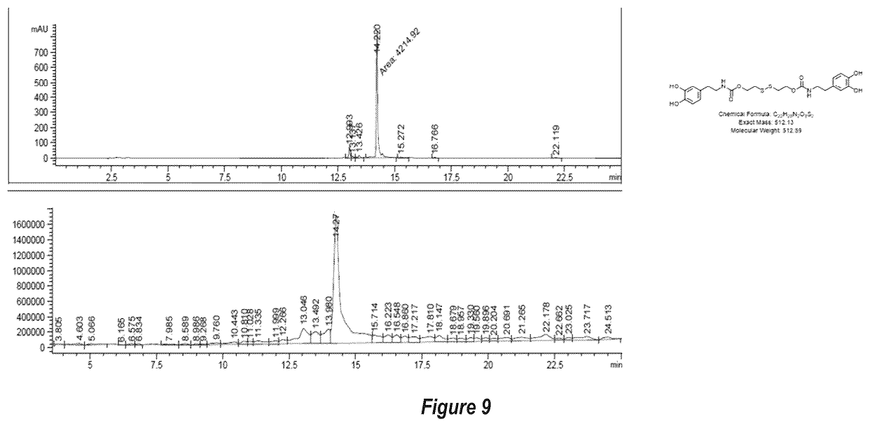

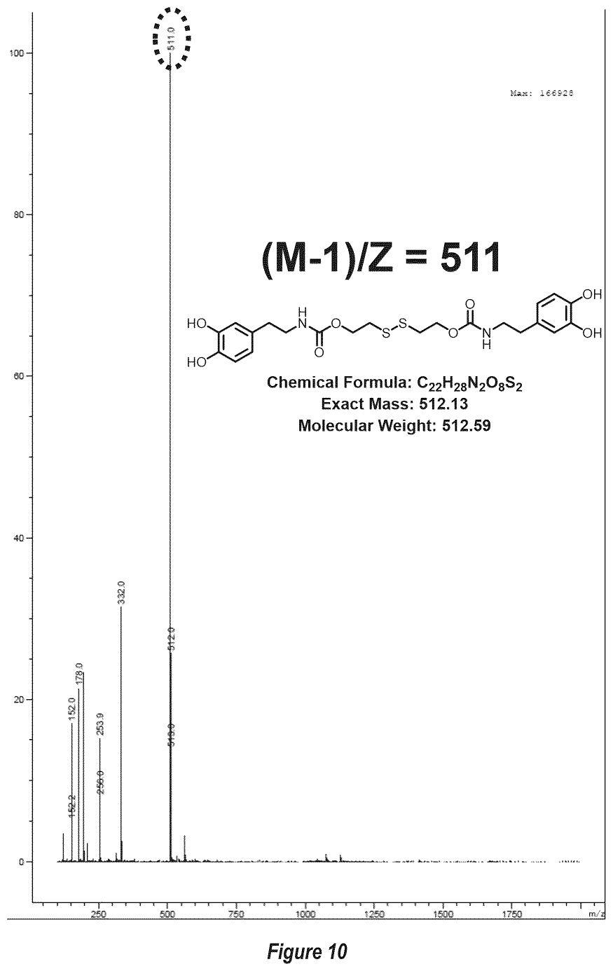

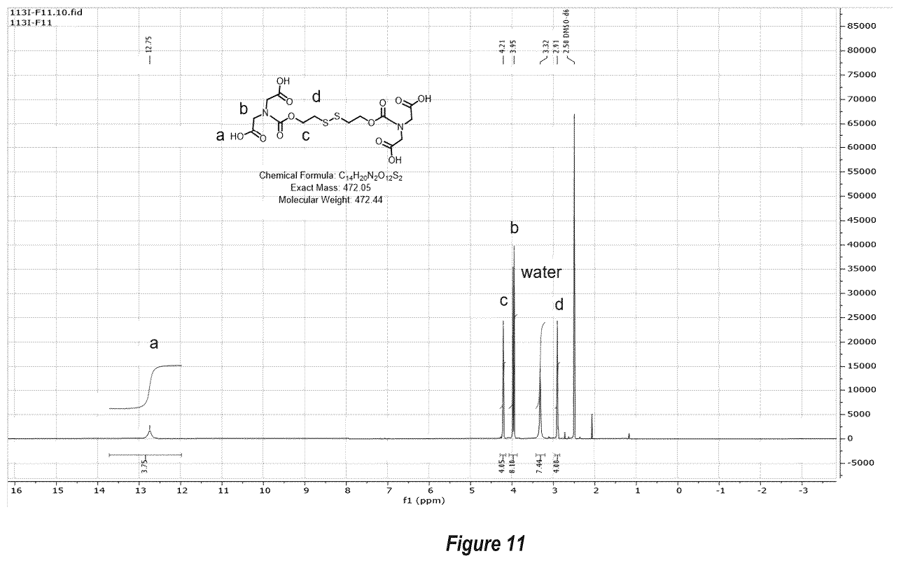

Relating to structure 1, FIG. 8 shows ¹H NMR data (400 MHZ, d6-DMSO) of disulfanediylbis(ethane-2,1-diyl) bis((3,4-dihydroxyphenethyl) carbamate) product. FIG. 9 shows HPLC of purified product and MS TIC of disulfanediylbis(ethane-2,1-diyl) bis((3,4-dihydroxyphenethyl) carbamate) chelator. FIG. 10 shows mass spectrum of TIC in product region showing M−1/Z of 511.

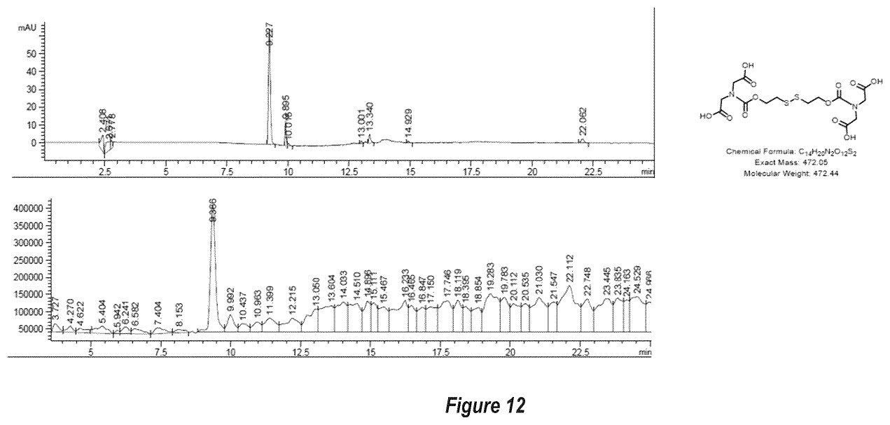

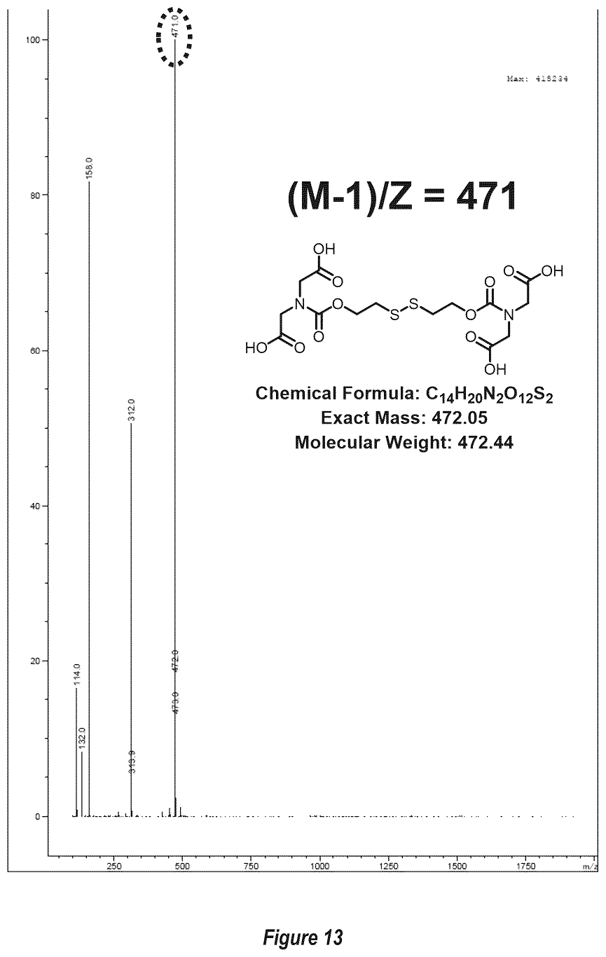

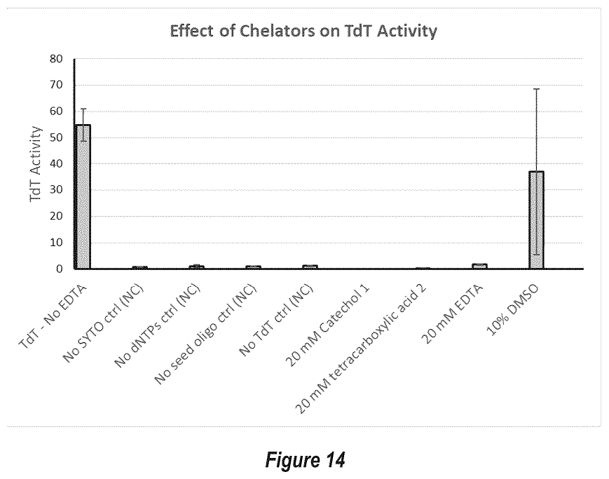

Relating to structure 2, FIG. 11 shows ¹H NMR data (400 MHZ, d6-DMSO) of 3,14-bis(carboxymethyl)-4,13-dioxo-5,12-dioxa-8,9-dithia-3,14-diazahexadecanedioic acid product in d6-DMSO. FIG. 12 shows HPLC of purified product and MS TIC of 3,14-bis(carboxymethyl)-4,13-dioxo-5,12-dioxa-8,9-dithia-3,14-diazahexadecanedioic acid. FIG. 13 shows mass spectrum in area of interest showing M−1/Z of 471.

The protocol for collecting HPLC data are described in International Application No. PCT/US2023/074179, the entire contents of which are incorporated herein by reference.

HPLC-MS (LC-MS) data were collected using an Agilent 1260 Infinity instrument with an Agilent 6120 Quadropole MS. Separations were performed using an Agilent Infinity Lab Poroshell EC-C18 column (4.6×100 mm, 2.7 μm) using the following solvent system at a flow rate of 0.5 mL/min: solvent A=0.05 M triethylammonium acetate (TEAA); solvent B=20% MeCN/0.05M TEAA; gradient method: 90% A/10% B for 3 min; linear gradient form 90% A/10% B to 20% A/80% B from 3-5 min; linear gradient from 20% A/80% B from 5 min to 8 min; maintained at 100% B from 8 min to 20 min. Compounds were detected by UV absorption at 210 nm, 254 nm, 280 nm, or 320 nm. Molecular weight range 400-2000; capillary voltage 3750 (pos) and 3500 (neg).

### Example 4. Inhibition of TdT Via Cleavable Metal Chelators

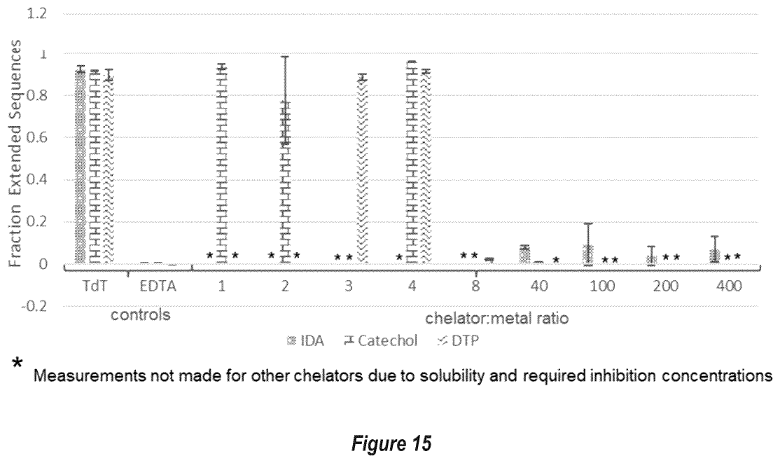

Preliminary experiments indicated that the new redox-mediated chelator compounds affect TdT mediation of DNA synthesis when used at similar concentrations as a canonical chelator EDTA. FIG. 14 provides data from an extension assay wherein wild-type TdT is incubated with a single-stranded DNA oligomer in the presence of free nucleotides and a DNA-intercalating dye (SYTO 13). As DNA synthesis occurs, fluorescent signal increases as more interactions of the intercalating dye with the ssDNA leads to higher fluorescent signal. Pre-incubation of metal cofactors (in this case, MnCl₂) with EDTA leads little-to-no-assayed ssDNA growth. The new compounds (catechol 1 and imidodiacetic acid 2) show similar activity in that no assayed ssDNA growth is observed.

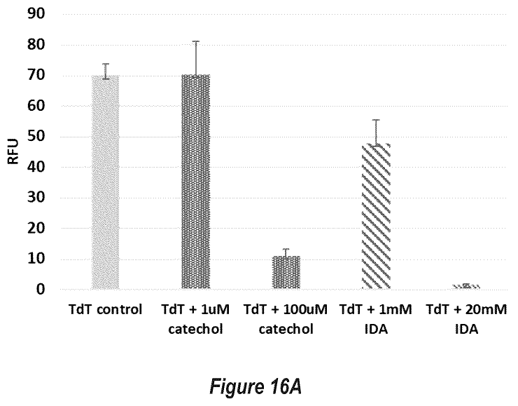

FIG. 15 shows results of a bead-based TdT extension assay using various chelator: metal ratios, 5 μM C7-C, and 0.250 mM Mn²⁺. When TdT is inhibited, as in the presence of EDTA, the fraction of extended sequences is markedly decreased. Extension assay was performed for 2 minutes at 37° C.

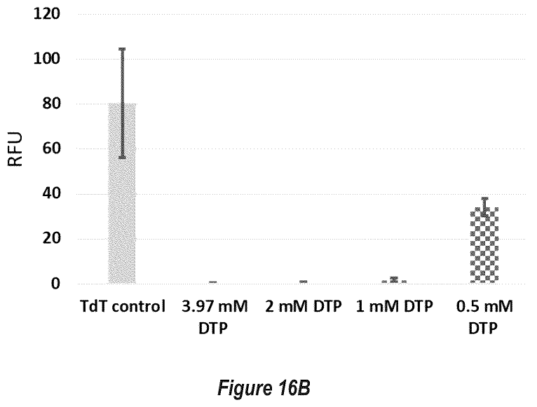

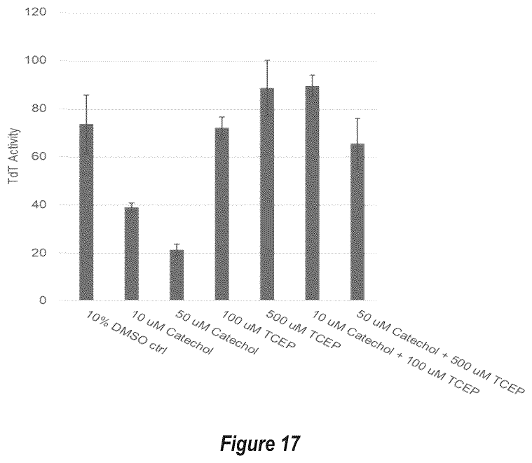

FIG. 16A shows a bead-based TdT extension assay using various concentrations of catechol 1 and tetraacid 2 (‘IDA’), 5 μM C7-C, and 0.250 mM Mn²⁺. FIG. 16A suggests inhibition dependence on feed ratio of metal to inhibitor and choice of chelator. Extension assay was performed for 2 minutes at 37° C. FIG. 16B shows a bead-based TdT extension assay using DTP, 5 μM C7-C, and 0.250 mM Mn²⁺. FIG. 16B suggests inhibition dependence on feed ratio of metal to inhibitor. Extension assay was performed for 2 minutes at 37° C.

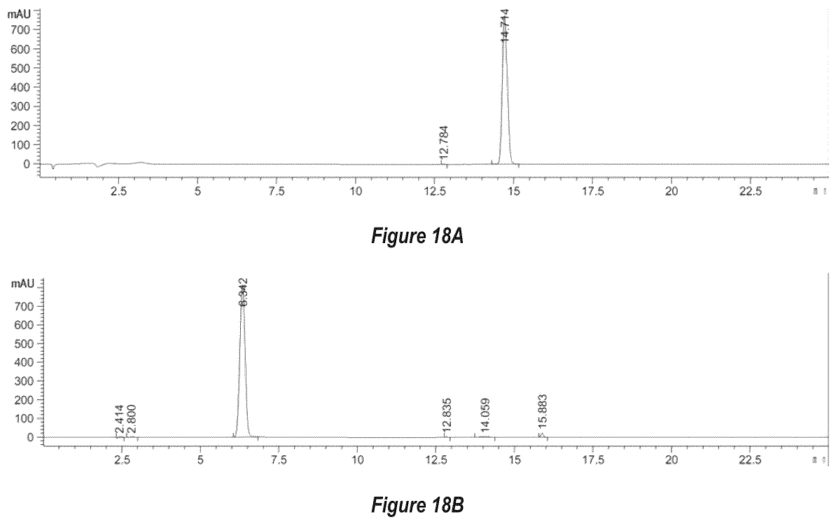

FIG. 17 shows TdT extension assay in the presence of a chelator (catechol 1), a reducing agent (TCEP), and the catechol pre-incubated with a reducing agent (TCEP). TCEP=tris(2-carboxyethyl) phosphine. With the TCEP reducing agent added, catechol 1 is cleaved and TdT activity is recovered.

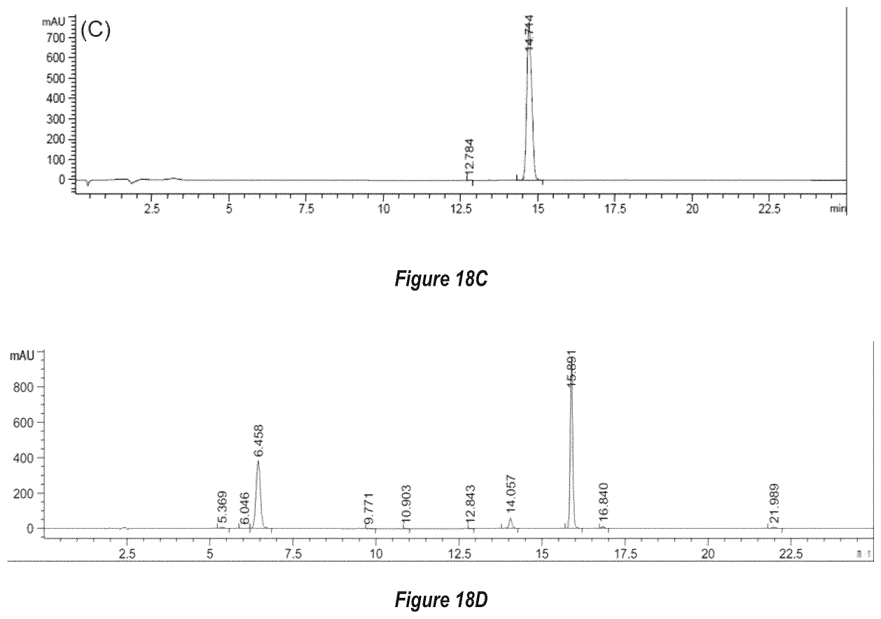

FIGS. 18A-18D show HPLC chromatograms of chelator solutions (FIG. 18A) DTP before bulk electrochemical reduction (FIG. 18B) 2-mercaptopyridine (degradation product) from commercial stock (FIG. 18C) DTP solution after 24 hours without bulk electrochemical reduction (FIG. 18D) DTP solution after 24 hours with bulk electrochemical reduction. Bulk reduction of DTP: A 50 mL solution was prepared by dissolving DTP (22 mg) and 3.7 mg MnCl₂ with 25 mL of milliQ water. Once dissolved, the solution was diluted with 25 mL of a stock 4× tris(hydroxymethyl)aminomethane (Tris)/NaCl solution (pH 7, 100 mM TRIS, 400 mM NaCl) bringing the total volume to 50 mL. The sample required heating for full dissolution. The resulting solution was a 2 mM DTP solution with 0.6 mM MnCl₂ in TRIS. Samples were aliquoted and used for specific testing. The samples that were exposed to electrochemistry using a Biologic VMP-300. The test setup included a Pt wire working electrode (1 mm OD×4 mm 1), a Pt wire counter electrode extended from coil (˜12 mm long), and a Ag/AgCl reference electrode. These electrodes were ported through a cap into a culture vial with a stir bar. the culture vial was cut to minimize head space and we used 7 ml for each samples. For the bulk reduction, in the three-electrode system, the solutions were exposed to −0.65V for 20 seconds, followed by an off voltage of 0.1V for 10 seconds. This process was repeated 2500 times. This led to approximately 1.63C of charge passing through the system.

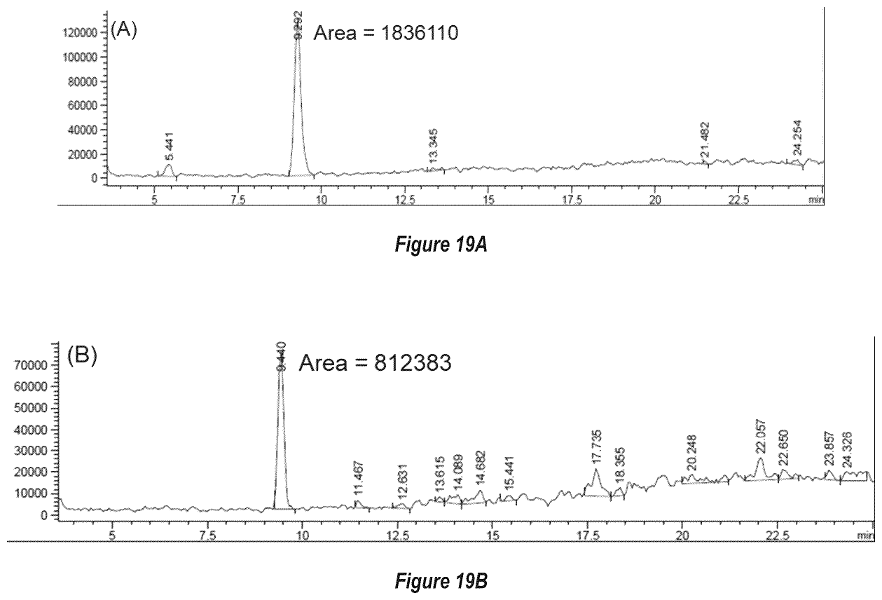

FIGS. 19A and 19B show HPLC-MS TIC of tetraacid 2 before (FIG. 19A) and after (FIG. 19B) bulk electrochemical reduction. Bulk reduction of tetraacid 2: A 10 mL solution was prepared by mixing 200 μL of 52 mM IDA with 1.25 mg MnCl₂ dissolved in 200 μL of miliQ water. the solution was then diluted with 4 mL of miliQ water before adding 2.25 4× TRIS, after addition, the pH was adjusted to 6.9 before diluting to the final 10 mL total volume. The resulting solution was a 1 mM IDA solution with 1 mM MnCl₂ in TRIS. Samples were aliquoted and used for specific testing. The samples that were exposed to electrochemistry using a Biologic VMP-300. The test setup included a Pt wire working electrode (1 mm OD×4 mm 1), a Pt wire counter electrode extended from coil (˜12 mm long), and a Ag/AgCl reference electrode. These electrodes were ported through a cap into a culture vial with a stir bar. the culture vial was cut to minimize head space and we used 7 ml for each samples. For the bulk reduction, in the three-electrode system, the solutions were exposed to −0.7V for 20 seconds, followed by an off voltage of 0V for 10 seconds. This process was repeated 7000 times. The samples before and after were measured by LCMS.

Protocol for bead-based TdT extension assay. The ability of chelators to inhibit stepwise addition of nucleotides via TdT-dNTP conjugates was tested using a bead based assay: Magnetic beads (Dynabeads MyOne streptavidin beads (C1)) were functionalized with biotinylated, double-stranded DNA (dsDNA) that contains a 3′ 30-base overhang, which serves as the seed oligo for extension. Beads were prepared in buffer (20 mM potassium phosphate, 100 mM NaCl, pH 6.5) and washed with 3 times with Superblock prior to use. TdT-dNTP conjugates were prepared at 10 μM concentrations in buffer solution (20 mM potassium phosphate buffer, 100 mM NaCl, pH 6.5). Chelators and MnCl₂ solutions were prepared at appropriate ratios (in water) so that final reaction concentrations were at 125 nM in MnCl₂ and incubated for 10 minutes at room temperature. Once incubation was complete, TdT-dNTP conjugates were added to the chelator/MnCl₂ solutions so that TdT-dNTP conjugates were at a final concentration of 5 μM. As controls, solutions with no chelator or with TCEP (for recovery activity) were also prepared.

Once all reagents were prepared, ds-DNA functionalized beads (10 μL volume) were added to low-bind Eppendorf tubes, followed by the TdT-dNTP conjugate/chelator/MnCl₂ solution (50 μL total reaction volume). The tubes were incubated at 37° C. for 2 min, then tubes were placed on ice for 1 min. The beads were washed by placing tubes on a magnetic tube rack three times with 1 mL of buffer (20 mM potassium phosphate, 100 mM NaCl, pH 6.5). To elute the ssDNA from the washed beads, the tubes were placed in the magnetic rack, and the supernatant was removed and replaced with 0.1 M NaOH (aq). After thorough mixing with a pipette and re-settling of the beads using the magnetic rack, the supernatant was transferred to a fresh low-bind tube, and 50 μL of 0.1 M HCl (aq) was added to neutralize the solution. The collected ssDNA was sequenced using the protocol reported in International Application No. PCT/US2023/074179.

Sequencing Protocol of ssDNA.

Sequencing sample preparation began with polyadenylation (for sequences expected to end in thymine, cytosine, or guanine) or the addition of a poly(T) tail (for sequences expected to end in adenine) to the 3′ end of each DNA sample using commercial TdT. The samples were then amplified using primers that bind to a conserved 5′ sequence and the complementary 3′ tail while incorporating Illumina sequencing priming sites into the synthesized amplicons. This step was performed using qPCR in order to achieve optimal amplification of all samples regardless of input DNA concentration. A portion of each PCR product was analyzed by gel electrophoresis to confirm the size and relative abundance of the amplicons. The products of all samples were then normalized based on relative abundance and used as template for a second PCR reaction, which incorporated Illumina flow cell adaptors and a variable index into each sample. The products of the final PCR reaction were characterized by gel electrophoresis and all indexed samples were pooled together proportionally based on relative abundance. Size selection and purification of the pooled library was performed by DNA gel extraction and the final library was analyzed by Qubit dsDNA HS assay and Tapestation D1000 screentape to determine the final molar concentration. The library was then diluted, denatured and sequenced following standard Illumina protocols.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of nucleic acid molecule synthesis comprising the steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker;
(c) providing at least one metal cofactor complexed to at least one cleavable chelating agent, wherein the chelating agent is capable of releasing the metal cofactor upon cleavage;
(d) selectively activating the TdT by cleaving the chelating agent;
(e) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand;
(f) exposing the TdT to conditions sufficient to deactivate it and remove it from the nucleic acid molecule; and
(g) repeating steps (b) through (f) thereby synthesizing a nucleic acid molecule.

2. The method of claim 1, wherein the chelating agent is cleavable by a reducing agent, an oxidizing agent, a redox-mediating agent, or a combination thereof.

3. The method of claim 1, wherein cleaving the chelating agent occurs through a voltage change at an electrode.

4. The method of claim 1, wherein the metal cofactor comprises iron, magnesium, manganese, cobalt, copper, zinc, molybdenum, or a combination thereof.

5. The method of claim 1, wherein the chelating agent comprises a cleavage site comprising a disulfide bond.

6. The method of claim 1, wherein the chelating agent comprises a catechol group.

7. The method of claim 1, wherein the chelating agent comprises an EDTA analog.

8. The method of claim 1, wherein the chelating agent comprises a tetracarboxylic acid group.

9. The method of claim 1, wherein the chelating agent comprises the compound of formula 1 or formula 2, shown as follows:

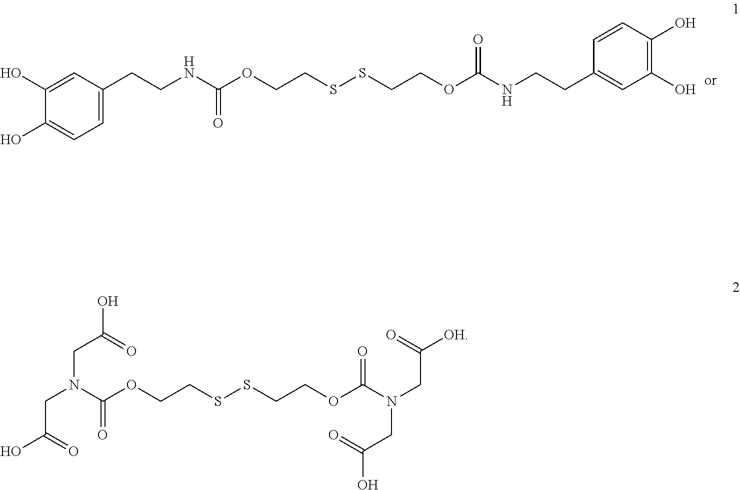 [SMILES: O=C(NCCc1ccc(O)c(O)c1)OCCSSCCOC(=O)NCCc1ccc(O)c(O)c1 ; O=C(O)CN(CC(=O)O)C(=O)OCCSSCCOC(=O)N(CC(=O)O)CC(=O)O]

10. The method of claim 1, wherein the conditions sufficient to deactivate the TdT and remove it from the nucleic acid molecule comprise digestion by an enzyme; chemical deactivation; electrochemical deactivation; photochemical deactivation; or a combination thereof.

11. A method of nucleic acid molecule synthesis in a buffer comprising the steps of:
(a) providing a single-stranded nucleic acid molecule comprising a 5′ and a 3′ end;
(b) providing an engineered terminal deoxynucleotidyl transferase (TdT), wherein the engineered TdT comprises a nucleotide molecule covalently attached to the TdT via a cleavable linker and at least one metal cofactor;
(c) contacting the TdT and the single-stranded nucleic acid molecule under conditions suitable for the TdT to bind to the 3′ end of the nucleic acid molecule and form a TdT-nucleic acid strand complex, thereby incorporating the nucleotide into the nucleic acid strand to create an extended nucleic acid strand;
(d) providing an inactivated pH sensitive enzyme, wherein the pH sensitive enzyme is inactivated by pH conditions of the buffer;
(e) activating the pH sensitive enzyme through a change in pH conditions in the buffer, wherein the activated pH sensitive enzyme digests the TdT, thereby removing the TdT from the extended nucleic acid strand;
(f) inactivating the pH sensitive enzyme through a change in pH conditions; and
(g) repeating steps (c) through (f) at least once thereby synthesizing a nucleic acid molecule.

12. The method of claim 11, wherein the pH sensitive enzyme is a protease or hydrolase.

13. The method of claim 11, wherein the pH sensitive enzyme is proteinase K.

14. The method of claim 11, wherein the change in pH conditions is caused by a change in voltage.

15. The method of claim 11, wherein the change in pH conditions is caused by addition of an acidic agent, basic agent, or a combination thereof to the buffer.

16. The method of claim 11, wherein a change in pH conditions is mediated by electrochemically reducing a pH mediator to convert it to a proton donor.

17. The method of claim 11, wherein the pH sensitive enzyme inactivation step comprises lowering the pH below 5 or raising the pH above 10, including optionally lowering the pH into a range from 3.5 to 5.

18. The method of claim 1, wherein the method of nucleic acid molecule synthesis occurs on a solid surface having multiple sites for nucleic acid molecule synthesis.

19. A system for nucleic acid synthesis,
wherein the system comprises:
an engineered TdT, wherein the engineered TdT comprises a nucleotide molecule covalently attached to the TdT via a cleavable linker;
at least one metal cofactor complexed to at least one cleavable chelating agent;
a redox shuttle solution; and
two or more electrodes on a surface; or
wherein the system comprises:
an engineered TdT, wherein the engineered TdT comprises a nucleotide molecule covalently attached to the TdT via a cleavable linker and at least one metal cofactor;
an inactivated pH sensitive enzyme;
a redox shuttle solution; and
two or more electrodes on a surface.

20. A compound represented by: 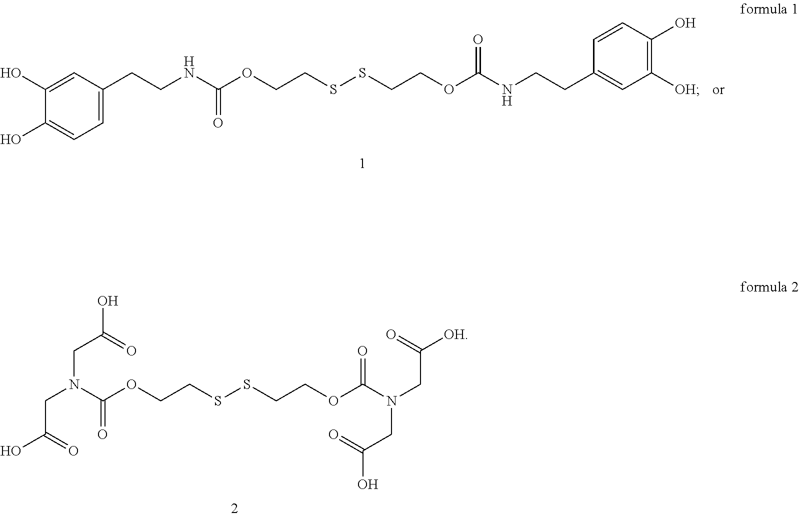 [SMILES: O=C(NCCc1ccc(O)c(O)c1)OCCSSCCOC(=O)NCCc1ccc(O)c(O)c1 ; O=C(O)CN(CC(=O)O)C(=O)OCCSSCCOC(=O)N(CC(=O)O)CC(=O)O]
